(19) United States
(12) Patent Application Publication
(10) Pub. No.: US 2025/0092421 A1
(43) Pub. Date: Mar. 20, 2025

(54) A MURINE CYTOMEGALOVIRUS VACCINE VECTOR FOR ADMINISTRATION IN A NON-MOUSE SUBJECT

(71) Applicants: Helmholtz-Zentrum für Infektionsforschung GmbH, Braunschweig (DE); University of Rijeka, Faculty of Medicine, Rijeka (HR)

(72) Inventors: Luka CICIN-SAIN, Braunschweig (DE); Kim YEONSU, Braunschweig (DE); Mario-Alberto YNGA-DURAND, Braunschweig (DE); Stipan JONJIC, Rijeka (HR)

(73) Assignees: Helmholtz-Zentrum für Infektionsforschung GmbH, Braunschweig (DE); University of Rijeka, Faculty of Medicine, Rijeka (HR)

(21) Appl. No.: 18/832,562

(22) PCT Filed: Jan. 25, 2023
(86) PCT No.: PCT/EP2023/051800

§ 371 Date: Jul. 24, 2024

(30) Foreign Application Priority Data
Jan. 25, 2022 (EP) 22153256.7
Apr. 28, 2022 (EP) 22170489.3

(51) Int. Cl. C12N 15/86; A61K 39/00; A61K 39/215; A61P 31/14

(52) U.S. Cl. CPC C12N 15/86; A61K 39/215; A61P 31/14; A61K 2039/51; A61K 2039/552; A61K 2039/572; A61K 2039/575; C12N 2710/16134; C12N 2710/16143; C12N 2770/20034

(57) ABSTRACT

The invention relates to a replication-deficient murine Cytomegalovirus (MCMV) vector for use in inducing an antigen-specific immune response in a subject, wherein the subject is not a mouse, and wherein said vector expresses a disease antigen. The invention further relates to a pharmaceutical composition comprising a replication-deficient murine Cytomegalovirus (MCMV) vector suitable to induce an antigen-specific immune response in a subject, wherein said vector expresses a disease antigen, and wherein the composition is configured for administration to a non-mouse subject. In embodiments, the vector has a disrupted immediate-early 2 (ie2) gene causing a replication deficiency of said vector in a non-mouse subject. The invention further relates to a pharmaceutical composition for use in inducing an antigen-specific immune response in a non-mouse subject to the expressed disease antigen.

The present invention is in the field of viral vectors and use thereof for inducing an antigen-specific immune response in a subject.

The invention relates to a replication-deficient murine Cytomegalovirus (MCMV) vector for use in inducing an antigen-specific immune response in a subject, wherein the subject is not a mouse, and wherein said vector expresses a disease antigen.

The invention further relates to a pharmaceutical composition comprising a replication-deficient murine Cytomegalovirus (MCMV) vector suitable to induce an antigen-specific immune response in a subject, wherein said vector expresses a disease antigen, and wherein the composition is configured for administration to a non-mouse subject. In embodiments, the vector has a disrupted immediate-early 2 (ie2) gene causing a replication deficiency of said vector in a non-mouse subject.

The invention further relates to a pharmaceutical composition for use in inducing an antigen-specific immune response in a non-mouse subject to the expressed disease antigen.

The invention further relates to a method for inducing an antigen-specific immune response in a subject, comprising administering a replication deficient murine Cytomegalovirus (MCMV) vector, wherein the subject is not a mouse, and wherein said vector expresses a disease antigen.

The invention further relates to a method for inducing an antigen-specific immune response in a subject, comprising administering a pharmaceutical composition comprising a replication-deficient murine Cytomegalovirus (MCMV) vector, wherein said vector expresses a disease antigen, and wherein the composition is configured for administration to a non-mouse subject, preferably comprising a human or veterinary acceptable carrier, excipient, vehicle and/or adjuvant.

### BACKGROUND OF THE INVENTION

The severe COVID-19 global pandemic has claimed millions of lives and resulted in severe economic disruption worldwide. Influenza pandemics have also resulted in global disruptions, such as the H1N1 Spanish flu in 1918, the H3N2 Hong Kong flu in 1968 and the H1N1dpm09 swine flu in 2009, and resulted in rapid global spread of this respiratory disease. In addition to these influenza pandemics, seasonal influenza epidemics regularly cause elevated morbidity and mortality in the colder seasons. Both influenza virus (IAV) and SARS COV-2 may cause mild to severe respiratory illnesses and pose a particular threat to at-risk groups, such as elderly people or people with pre-existing medical conditions. Both of these respiratory viruses depend on a viral surface protein for attachment and entry into host cells. In the case of IAV, viral hemagglutinin (HA) is the major surface glycoprotein required for cell entry. Likewise, SARS-COV-2 uses the spike protein(S) to bind its cellular receptor ACE2 and to drive membrane fusion during virus entry. Therefore, SARS-COV-2 S and IAV HA are the main antigenic targets in vaccine formulations against these viruses.

There are more than 200 vaccine projects targeting SARS-COV-2 using formulations that include viral proteins, viral vector vaccines, and mRNA vaccines. Some of these vaccines have already been approved for use in humans or are in advanced clinical trials with promising results.

However, all of the candidates raise safety concerns due to side effects such as fever, fatigue and headache, and most vaccines (or vaccine candidates) require a prime/boost vaccination protocol at multiple-week intervals, raising issues of delivery logistics and compliance. Although mRNA vaccines show great promise in the context of the COVID-19 pandemic, experience with their use in clinical settings remains limited.

Vaccines against influenza target the predicted prevailing strains in each upcoming flu season and are especially recommended for people at high risk, such as children, elderly individuals and immunocompromised individuals. While influenza vaccines are available, their efficacy is approximately 19-60% depending on the flu season.

WO 2011/093858 discloses that a recombinant replication-deficient murine cytomegalovirus (MCMV) induces a T cell-based immune response against a heterologous antigen expressed by the CMV in a subject.

Klyushnenkova et al. discloses that a murine CMV-based vaccine expressing a human prostate specific antigen induces an anti-tumor immune response (Elena et al, “A Cytomegalovirus-based Vaccine Expressing a Single Tumor-specific CD8 (+) T-cell Epitope Delays Tumor Growth in a Murine Model of Prostate Cancer”, Journal of Immunotherapy, 1 Jun. 2012). The anti-tumor effect was demonstrated in a humanized double-transgenic mouse model. The ie2 gene can be deleted or mutationally modified without affecting virus replication in vivo.

Mohr et al. discloses that a spread-deficient murine CMV with a deleted essential gene M94 induces a robust CD4+ and CD8+ T-cell response and a neutralizing antibody response (Mohr et al, “A Spread-Deficient Cytomegalovirus for Assessment of First-Target Cells in Vaccination”, Journal of Virology, 1 Aug. 2010). MCMV-ΔM94 does not revert to replication-competent virus in the mouse.

The provision of effective vaccines and vaccine vectors remains a serious medical need. Vaccines based on viral vectors, such as adenovirus-based vaccines, provide robust immune protection and are part of the vaccine portfolio against the current COVID-19 pandemic, but cannot be efficiently re-used against other targets. This limitation is due to vector specific neutralizing antibodies, which are elicited during the initial vaccine administration. Hence, on the one hand, this limits antigen delivery to cells upon repeat administration. On other hand, it facilitates the induction of recall immune responses against adenovirus antigens at the expense of immune priming.

Therefore, additional viral vectors that elicit robust immune responses and protection are in urgent need not only for COVID-19 and Influenza, but also other infectious diseases caused by virus, bacteria and/or parasites. In particular, vectors are desired that elicit minimal or no disease manifestations during administration yet elicit robust and lasting immunity against encoded antigens.

### SUMMARY OF THE INVENTION

In light of the prior art the technical problem underlying the present invention is to provide alternative and/or improved means for inducing an antigen-specific immune response in a subject.

This problem is solved by the features of the independent claims. Preferred embodiments of the present invention are provided by dependent claims.

The invention therefore relates to a replication-deficient murine Cytomegalovirus (MCMV) vector for use in inducing an antigen-specific immune response in a subject, wherein the subject is not a mouse, and wherein said vector expresses a disease antigen.

The invention also relates to a replication-deficient murine Cytomegalovirus (MCMV) vector for use as a medicament in inducing an antigen-specific immune response in a subject, for example to induce immunity to said antigen, wherein the subject is not a mouse, and wherein said vector expresses a disease antigen.

The invention further relates to a pharmaceutical composition comprising a replication-deficient murine Cytomegalovirus (MCMV) vector suitable to induce an antigen-specific immune response in a subject, wherein said vector expresses a disease antigen, and wherein the composition is configured for administration to a non-mouse subject.

A further aspect relates to a method for inducing an antigen-specific immune response in a subject, comprising administering a replication deficient murine Cytomegalovirus (MCMV) vector, wherein the subject is not a mouse, and wherein said vector expresses a disease antigen. A further aspect relates to a method for inducing an antigen-specific immune response in a subject, comprising administering a pharmaceutical composition comprising a replication-deficient murine Cytomegalovirus (MCMV) vector, wherein said vector expresses a disease antigen, and wherein the composition is configured for administration to a non-mouse subject, preferably comprising a human or veterinary acceptable carrier, excipient, vehicle and/or adjuvant.

In embodiments, the vector has a disrupted immediate-early 2 (ie2) gene causing a replication deficiency of said vector in a non-mouse subject.

Vaccines based on viral vectors are widely used in experimental and clinical conditions. On one hand, several vectors based on adenoviral viruses have been approved in numerous countries for clinical use against COVID-19, including Vaxzevria, COVID-19 Vaccine Jannsen, Convidecia or Sputnik-V, either as a single shot or as a prime-boost formulation. Other examples of vaccine vectors with limited use in veterinary or human medicine include Newcastle disease virus (NDV), Vesicular stomatitis virus (VSV) or modified vaccinia Ankara (MVA) vectors. These viruses were tested in experimental conditions as vectors for MERS, Ebola, Rabies, influenza, West-Nile virus and many other antigens. They have shown solid biosafety profiles, the ability to elicit T-cell responses and provide immune protection.

CMV vectors were shown to provide robust and lasting immunogenicity upon immunization, both based on cellular or humoral immunity. Rhesus CMV vectors were used in the rhesus models of tuberculosis, AIDS or malaria infection. Murine (mouse) CMV vectors were used against influenza, tumors, herpes simples, tuberculosis, Ebola, in the mouse models of infection. A human CMV vector is currently in phase I clinical trials.

Most non-CMV based models provide in general a cellular and humoral immune response that decreases over time. The CMV vectors are unique in their ability to elicit a life-long protective immunity, due to the vector's ability to episomally maintain its latent genome in the cells of the vaccinated host and re-stimulate the immune system over time.

The adenoviruses possess some limited ability to persist in the host cells for a while longer, and thus induce responses that last somewhat longer. However, the immunity against the adenoviral vector limits its ability to be reused against independent infectious targets. Furthermore, natural immunity against the human adenovirus vectors limits their usage. Hence, CMV-based vectors offer discrete advantages over the non-CMV ones.

Among the CMV vectors, numerous studies have focused on properties of the MCMV vector in the mouse model, but to the knowledge of the inventors have not tested its functionality in a species that is not the natural host.

Although WO 2011/093858 teaches generating a long term, repeatedly stimulated immune response against a heterologous antigen in a subject, by administering a recombinant, replication-deficient cytomegalovirus expressing a heterologous antigen, no experiments are conducted using a MCMV in a human subject. Instead, Example 6 of the application discloses that human replication-deficient CMV expressing tumor antigens was used in phase I clinical trials to patients for example with stage 4 melanoma. To the knowledge of the inventors, no previous disclosure of the prior art teaches administration of murine CMV in a non-murine host, preferably humans, to induce an antigen-specific immune response in the host against a (transgenic expressed) disease antigen.

Likewise, numerous studies showed the efficacy of rhesus CMV (RhCMV) vaccine vectors in rhesus monkeys, the natural host species. Vectors based on such approaches would require that the human CMV genome (HCMV) is used as the vaccine vector. Such vectors exist and are in early clinical trials. However, collectively these vectors have several limitations: The HCMV is a pathogenic virus that may cause harm to the human host and use of HCMV based vectors comes with inherent risks for pathogenesis. Such risks may be mitigated by genetic modifications of the virus, including HCMV vector growth attenuation by gene deletions in recombinant DNA approaches or insertion of sequences that destabilize essential viral proteins.

In contrast, the vector of the present invention is inherently safer in human cells, because the MCMV is poorly adapted to grow in them.

The exquisite adaptation of the CMV to its natural host species results in robust repression of immune sensing and thus of the adjuvant potential of the CMV vectors. Our vector system activates the innate immune responses.

The natural immunity to HCMV may interfere with the ability of an HCMV vector to express antigen in the host. HCMV is ubiquitous and the majority of the human population is seropositive to this virus. The MCMV does not naturally infect people, and hence this concern is of no consequence.

The potential for homologous recombination of HCMV vaccine vector with naturally occurring wild-type HCMVs is stronger than for MCMV vectors, whose genetic homology to HCMV is comparably modest.

Evidence on the use of MCMV vectors in heterologous species is very restricted. One previous publication (Wang et al. J Virol 2003; 77 (13): 7182-92) showed that infection of human dendritic cells with an MCMV vector that encoded the gp120 gene of HIV elicited cellular immune responses to the antigen in vitro. However, that study was based on a vector that was replication competent and expressed the ie2 gene and in human retinal pigment epithelium. Moreover, the authors of this study did not test the ability of their vector to induce immune responses and immune protection in an in vivo system.

In embodiments, the present invention thus utilizes a replication deficient MCMV vector, preferably an ie2-deficient MCMV, as a vaccine vector that is replication restricted in all tested human cell types but fully replication competent in murine cells. The vector is capable of expressing antigens in human cells, and immunogenic and protective in an animal model that is not the natural host species of MCMV.

To the knowledge of the inventors, there are no publications on vaccine vectors that are based on an MCMV genetic platform, and that are safe to be broadly used in veterinary and human medicine applications. In the examples we demonstrate that the ie2 gene, known to be dispensable for MCMV growth in murine cells, is essential for MCMV replication in human retinal antigen epithelium (RPE) cells, that MCMVs lacking ie2 can still express genes and antigen in human cells, in particular in the professional antigen presenting cells, and that this allows antigen recognition by antigen-specific T-lymphocytes, and that such vectors elicit humoral immune responses and protection in a nonmurine vaccinated host, on the paradigmatic example of a hamster vaccination and challenge with SARS-COV-2.

The various aspects of the invention are unified by, benefit from, are based on and/or are linked by the common and surprising finding that a replication-deficient murine Cytomegalovirus (MCMV) vector can be effectively used in inducing an antigen-specific immune response in a subject that is not the native host of the viral vector.

The application of viral vectors in non-natural hosts represents an unexpected finding of the present invention. To the knowledge of the inventors, the use of e.g. a murine CMV vector in human subjects has been neither disclosed nor suggested in the art. The medical use of the invention thus represents a novel approach towards providing safe and effective viral vectors in human and veterinary settings.

Human CMV viral vectors are known as vaccine vehicles for the expression of disease antigens in human subjects. The present invention departs from this use of a vector in its natural host by electing a non-human CMV vector for administration in e.g. humans. The replication deficiency of the vector of the present invention is effective in the subject for administration but does not necessarily hamper replication of the vector in a mouse host. In other words, the vector of the invention may replicate and thus be produced in large amounts in the natural host of the vector, but does not replicate in e.g. a human subject.

Such a replication-deficient murine cytomegalovirus vector is able to induce an antigen-specific immune response against infectious disease or cancer in a host without replicating and thus without spreading in the host. This advantage satisfies the urgent medical need for an efficient vaccine. So far, vaccines based on viral vectors, such as adenovirus-based vaccines, provide a robust immune protection and part of the vaccine portfolio against the current COVID-19 pandemic, but cannot be efficiently re-used against other targets. This limitation is due to vector specific neutralizing antibodies, which are elicited during the initial vaccine administration. This unwanted immune response limits the antigen delivery to cells upon repeat administration and facilitates the induction of recall immune response against adenovirus antigen at the expense of immune priming.

One advantage of the present invention is based on the surprising finding that the contemplated viral vector is replication deficient in the non-mouse subject and thus will not spread in the host, nor elicit immune priming in the host raised by viral vector per se. Meanwhile, a robust immune protection can be obtained against a wide range of infectious diseases and cancers depending on selection of the disease antigen.

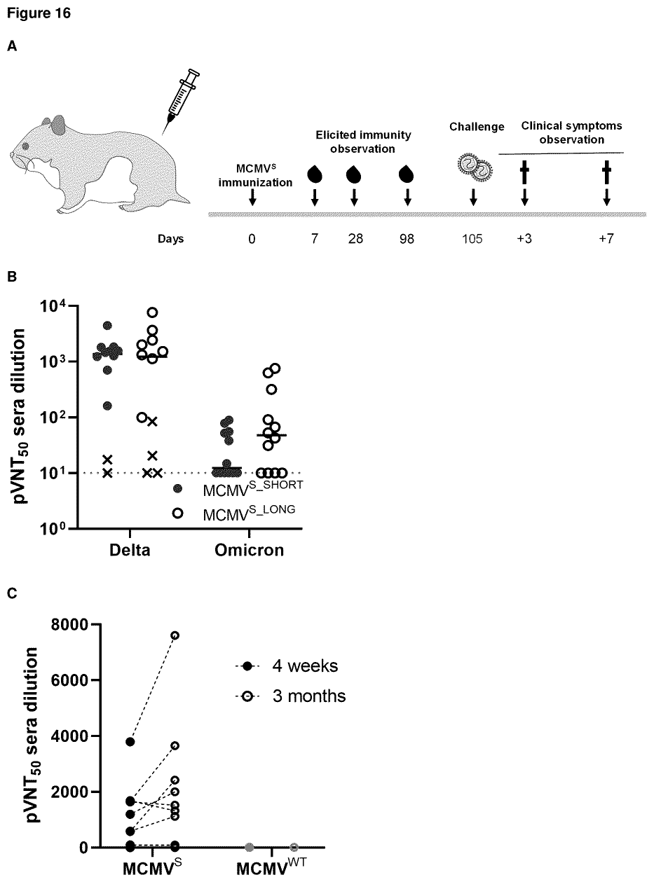

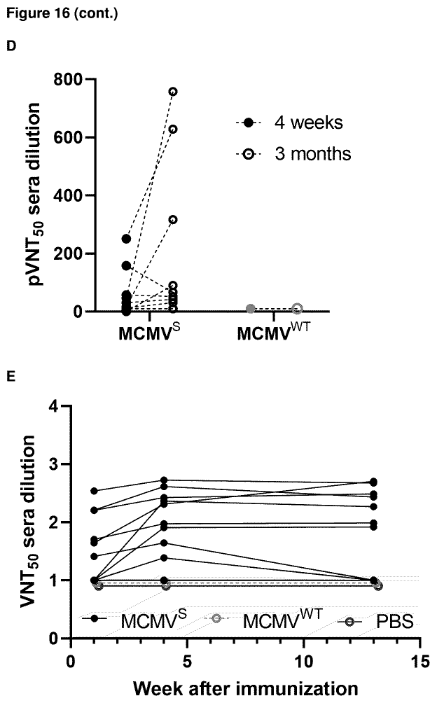

Furthermore, as can be derived from FIG. 16, in particular FIGS. 16 C and D, the pVNT50 values of individual hamsters 4 weeks or 3 months after vaccination against different SARS-COV-2 variants show that increased antibody titers are obtained over time. This represents a beneficial and unexpected result, indicating that antigen-specific immunity may, after administration of the vaccine mCMV vector, not wane or reduce over time, thus reducing the need for booster immunizations, as is the case for e.g., mRNA vaccines.

In embodiments, the vector as described herein is characterized in that an open reading frame for the disease antigen is expressed by an immediate-early MCMV promoter.

In embodiments, an open reading frame for the disease antigen is expressed by a constitutive promoter or an inducible promoter. In embodiments, the constitutive and inducible promoters can be an exogenous promoter operably linked to an open reading frame for the disease antigen which drives the expression of the disease antigen in the host.

The constitutive promoters known in the art include, but are not limited to, a CAG promoter, short variant of CMV early enhancer/chicken β actin (sCAG) promoter, hCMV promoter, a phosphoglycerate kinase (PGK) promoter, a human synapsin promoter (hSYN), the SV40 early (SV40e) promoter region, the promoter contained in the 3′ long terminal repeat (LTR) of Rous sarcoma virus (RSV), the promoters of the E1A or major late promoter (MLP) genes of adenoviruses (Ad), the cytomegalovirus (CMV) early promoter, the herpes simplex virus (HSV) thymidine kinase (TK) promoter, a baculovirus IE1 promoter, an elongation factor 1 alpha (EF1) promoter, a ubiquitin (Ubc) promoter, an albumin promoter, the regulatory sequences of the mouse metallothionein-L promoter and transcriptional control regions, the ubiquitous promoters (HPRT, vimentin, α-actin, tubulin and the like), the promoters of the intermediate filaments (desmin, neurofilaments, keratin, GFAP, and the like), the promoters of therapeutic genes (of the MDR, CFTR or factor VIII type, and the like), pathogenesis or disease related-promoters, and promoters that exhibit tissue specificity and have been utilized in transgenic animals, such as the elastase I gene control region which is active in pancreatic acinar cells; insulin gene control region active in pancreatic beta cells, immunoglobulin gene control region active in lymphoid cells, mouse mammary tumor virus control region active in testicular, breast, lymphoid and mast cells; albumin gene, Apo AI and Apo AII control regions active in liver, alpha-fetoprotein gene control region active in liver, alpha 1-antitrypsin gene control region active in the liver, beta-globin gene control region active in myeloid cells, myelin basic protein gene control region active in oligodendrocyte cells in the brain, myosin light chain-2 gene control region active in skeletal muscle, and gonadotropic releasing hormone gene control region active in the hypothalamus, pyruvate kinase promoter, villin promoter, promoter of the fatty acid binding intestinal protein, promoter of the smooth muscle cell α-actin, and the like. In addition, these expression sequences may be modified by addition of enhancer or regulatory sequences and the like. In embodiments, the vector as described herein is characterized in that the open reading frame for the disease antigen is inserted in the vector to replace any non-essential viral genomic sequences.

In the context of the present invention, an inducible promoter refers to regulatable promoter which is active only in response to specific stimuli, including e.g., exogenous stimuli, in specific tissues, in specific tumors, or after therapy.

In embodiments, an inducible promoter can be Tetracycline or Doxycycline inducible promoter. An inducible promoter can also be a tissue specific promoter and includes but is not limited to
leukocyte specific: CD11a promoter, CD11b promoter, CD18 promoter,
erythroid cells specific: β-Globin promoter/LCR,
B-lymphoma specific: immunoglobulin promoters,
Hepatocyte specific: PEPCK promoter, Albumin promoter, hAAT promoter,
Mammary carcinoma specific: MMTV-LTR, WAP promoter, β-casein,
Bronchiolar and alveolar epithelium specific: SPC promoter, SP-A and SP-B,
Undifferentiated myogenic cells specific: MCK promoter,
Myoblast specific: VLC1 promoter,
Lymphocytes specific: HIV-LTR.

An inducible promoter can further be a response element including but not limited to Tat/Rev-responsive elements and Tat-inducible element for CD4 positive cells. An inducible promoter can further be a cancer specific promoter CEA promoter for colon- and lung carcinomas, AFP promoter for hepatocellular carcinomas, SLPI promoter for carcinomas, thyrosinase promoter for melanomas, c-erbB2 promoter for breast, pancreatic, gastric carcinomas and lung cancer. An inducible promoter can further refer to a therapy inducible promoter including but not limited to Erg-1 promoter for irradiated tumors, Grp78 promoter for anoxic, acidic tumor tissue, MDR1 promoter for tumor treated with chemotherapy, HSP70 promoter for tumors treated with hyperthermy. A suitable promoter for driving the expression of a disease antigen can be decided by a skilled person in the art according to e.g. the aim of the delivery of contemplated MCMV to the host, the host per se and the delivery route.

In embodiments, the vector as described herein comprises a disrupted immediate-early 2 (ie2) gene, causing a replication deficiency of said vector in a non-mouse subject.

In one embodiment, the ie2 gene sequence is according SEQ ID NO:1:

ccgtgctgattcatatgccatatgagtgtattagggggctttccgcttgg
gaaattgggtaaaaagtccccgtattactcacatagggggcgtttggctt
tgcaaattaggggatttcagtgcatttggcattaaaaactattggttcta
gtcataaaacgggcggagttAACCATATAAAAGCTGTCCCCCATGCCATT
CGAGCCCAGAGCAGGACGGACCGCGGCTCGATACGACCCTATCTACGTTa
acgagcgggtgaacggagccccccaggccgggtaaggttgcattgggctc
ggctatgggacggggatttggcccggtactttaatatgggtggggtct

In one embodiment, deletion of the sequence in capitals (SEQ ID NO 3) causes a dysfunctional IE2.

SEQ ID NO 3:
AACCATATAAAAGCTGTCCCCCATGCCATTCGAGCCCAGAGCAGGACGGA
CCGCGGCTCGATACGACCCTATCTACGTT

The underlined sequence is exon 1 of the ie2 gene, according to SEQ ID NO 4:

GCCCAGAGCAGGACGGACCGCGGCTCGATACGACCCTATCTACGTT

Deletion of SEQ ID NO 3 represents merely an example for generating functional disrupted IE2. The skilled person can make a disrupted ie2 gene according to various means, as known by a skilled person in the art.

This murine ie2 is dispensable for growth and latency of MCMV vector in mice, but is indispensable for growth in a non-mouse subject. In embodiments, a disrupted ie2 gene causes a replication deficiency in human or other non-mouse subject due to its replication incompetency or replication inability in these subjects. This surprising and beneficial finding renders a suitable use of the vector as described herein in a non-mouse subject for inducing an antigen specific immune response without eliciting the immune priming against the viral vector.

In embodiments, the ie2 gene is disrupted by deletion of the ie2 gene or part thereof.

In a preferred embodiment, the ie2 gene is disrupted by deletion of a TATA box or deletion of the ie2 open reading frame or part thereof.

The TATA box of the ie2 gene is shown in bold type capitals, within SEQ ID NO 1 and 3.

In other embodiments, a replication deficient MCMV comprises a gene disruption causing replication incompetency or deficiency other than or additional to ie2 disruption. The contemplated disrupted gene can be any one or more genes in MCMV targeting conserved cellular functions. The mutation of these genes leads to a replication solely in mouse cells and replication incompetency or deficiency in non-mouse subject. These genes include but are not limited to viral genes targeting apoptotic molecules e.g., M36, M38.5, m41, those affecting promyelocytic leukemia (PML) associated nuclear bodies (e.g. IE1) or interferon signaling (e.g. M35, M27). The skilled person in the art can identify such genes and can identify the replication deficiency resulting from the disruption of such genes according to common skills in the art.

In embodiments, an open reading frame for the disease antigen is inserted in the vector to replace the ie2 open reading frame or part thereof.

In other embodiments, the disease antigen can be inserted into any location of the viral vector where the disease antigen can be under control of operably linked sequence, preferably a promoter driving the expression of the disease antigen.

In some embodiments, the operably linked sequence is a promoter including an inducible promoter. In embodiments, a reading frame for the disease antigen driven by an immediate-early MCMV promoter is inserted in the vector to replace any non-essential viral genomic sequences.

In one embodiment, the subject is human. In some embodiments, the subject is a non-human animal. Both human medical and veterinary applications are envisaged.

In embodiments, the immune response comprises a neutralizing antibody response or a T-cell response against the disease antigen.

In embodiments, the immune response comprises priming, maintaining and/or boosting the immune system of the subject. In embodiments, the priming, maintaining and boosting of the immune system can occur in a subject upon single or multiple delivery of the viral vector to the subject.

In embodiments, the disease antigen comprises an antigen or fragment(s) thereof of a pathogenic virus, bacteria, parasite and/or cancer cell.

In embodiments, the viral antigen comprises but is not limited to an antigen from a coronavirus, influenza virus, herpes virus, polyoma virus, papilloma virus, orthomyxovirus, paramyxovirus, picornavirus, parvovirus, reovirus, rhabdovirus, togavirus, African swine fever, adenovirus, coxsackie, arenavirus, a retrovirus, alfavirus, pestivirus, a flavivirus or an arbovirus.

In embodiments, the viral antigen is a Spike, Envelope, Membrane, Nucleoprotein of SARS-COV-2 or any part thereof.

In one embodiment, the nucleic acid sequence encoding a spike protein of the SARS-COV-2 is according the SEQ ID NO:2:

ATGTTCGTGTTTCTGGTGCTGCTGCCTCTGGTGTCCAGCCAGTGTGTGAACCTGACCACAAG
AACCCAGCTGCCTCCAGCCTACACCAACAGCTTTACCAGAGGCGTGTACTACCCCGACAAGG
TGTTCAGATCCAGCGTGCTGCACTCTACCCAGGACCTGTTCCTGCCTTTCTTCAGCAACGTG
ACCTGGTTCCACGCCATCCACGTGTCCGGCACCAATGGCACCAAGAGATTCGACAACCCCGT
GCTGCCCTTCAACGACGGGGTGTACTTTGCCAGCACCGAGAAGTCCAACATCATCAGAGGCT
GGATCTTCGGCACCACACTGGACAGCAAGACCCAGAGCCTGCTGATCGTGAACAACGCCACC
AACGTGGTCATCAAAGTGTGCGAGTTCCAGTTCTGCAACGACCCCTTCCTGGGCGTCTACTA
TCACAAGAACAACAAGAGCTGGATGGAAAGCGAGTTCCGGGTGTACAGCAGCGCCAACAACT
GCACCTTCGAGTACGTGTCCCAGCCTTTCCTGATGGACCTGGAAGGCAAGCAGGGCAACTTC
AAGAACCTGCGCGAGTTCGTGTTCAAGAACATCGACGGCTACTTCAAGATCTACAGCAAGCA
CACCCCTATCAACCTCGTGCGGGATCTGCCTCAGGGCTTCTCTGCTCTGGAACCCCTGGTGG
ATCTGCCCATCGGCATCAACATCACCCGGTTTCAGACACTGCTGGCCCTGCACAGAAGCTAC
CTGACACCTGGCGATAGCAGCAGCGGATGGACAGCTGGTGCCGCCGCTTACTATGTGGGCTA
CCTGCAGCCTAGAACCTTCCTGCTGAAGTACAACGAGAACGGCACCATCACCGACGCCGTGG
ATTGTGCCCTTGATCCTCTGAGCGAGACAAAGTGCACCCTGAAGTCCTTCACCGTGGAAAAG
GGCATCTACCAGACCAGCAACTTCCGGGTGCAGCCCACCGAATCCATCGTGCGGTTCCCCAA
TATCACCAATCTGTGCCCCTTCGGCGAGGTGTTCAATGCCACCAGATTCGCCTCTGTGTACG
CCTGGAACCGGAAGCGGATCAGCAATTGCGTGGCCGACTACTCCGTGCTGTACAACTCCGCC
AGCTTCAGCACCTTCAAGTGCTACGGCGTGTCCCCTACCAAGCTGAACGACCTGTGCTTCAC
AAACGTGTACGCCGACAGCTTCGTGATCCGGGGAGATGAAGTGCGGCAGATTGCCCCTGGAC
AGACAGGCAAGATCGCCGACTACAACTACAAGCTGCCCGACGACTTCACCGGCTGTGTGATT
GCCTGGAACAGCAACAACCTGGACTCCAAAGTCGGCGGCAACTACAATTACCTGTACCGGCT
GTTCCGGAAGTCCAATCTGAAGCCCTTCGAGCGGGACATCTCCACCGAGATCTATCAGGCCG
GCAGCACCCCTTGTAACGGCGTGGAAGGCTTCAACTGCTACTTCCCACTGCAGTCCTACGGC
TTTCAGCCCACAAATGGCGTGGGCTATCAGCCCTACAGAGTGGTGGTGCTGAGCTTCGAACT
GCTGCATGCCCCTGCCACAGTGTGCGGCCCTAAGAAAAGCACCAATCTCGTGAAGAACAAAT
GCGTGAACTTCAACTTCAACGGCCTGACCGGCACCGGCGTGCTGACAGAGAGCAACAAGAAG
TTCCTGCCATTCCAGCAGTTTGGCCGGGATATCGCCGATACCACAGACGCCGTTAGAGATCC
CCAGACACTGGAAATCCTGGACATCACCCCTTGCAGCTTCGGCGGAGTGTCTGTGATCACCC
CTGGCACCAACACCAGCAATCAGGTGGCAGTGCTGTACCAGGACGTGAACTGTACCGAAGTG
CCCGTGGCCATTCACGCCGATCAGCTGACACCTACATGGCGGGTGTACTCCACCGGCAGCAA
TGTGTTTCAGACCAGAGCCGGCTGTCTGATCGGAGCCGAGCACGTGAACAATAGCTACGAGT
GCGACATCCCCATCGGCGCTGGCATCTGTGCCAGCTACCAGACACAGACAAACAGCCCCAGA
CGGGCCAGATCTGTGGCCAGCCAGAGCATCATTGCCTACACAATGTCTCTGGGCGCCGAGAA
CAGCGTGGCCTACTCCAACAACTCTATCGCTATCCCCACCAACTTCACCATCAGCGTGACCA
CAGAGATCCTGCCTGTGTCCATGACCAAGACCAGCGTGGACTGCACCATGTACATCTGCGGC
GATTCCACCGAGTGCTCCAACCTGCTGCTGCAGTACGGCAGCTTCTGCACCCAGCTGAATAG
AGCCCTGACAGGGATCGCCGTGGAACAGGACAAGAACACCCAAGAGGTGTTCGCCCAAGTGA
AGCAGATCTACAAGACCCCTCCTATCAAGGACTTCGGCGGCTTCAATTTCAGCCAGATTCTG
CCCGATCCTAGCAAGCCCAGCAAGCGGAGCTTCATCGAGGACCTGCTGTTCAACAAAGTGAC
ACTGGCCGACGCCGGCTTCATCAAGCAGTATGGCGATTGTCTGGGCGACATTGCCGCCAGGG
ATCTGATTTGCGCCCAGAAGTTTAACGGACTGACAGTGCTGCCACCACTGCTGACCGATGAG
ATGATCGCCCAGTACACATCTGCCCTGCTGGCCGGCACAATCACAAGCGGCTGGACATTTGG
AGCTGGCGCCGCTCTGCAGATCCCCTTTGCTATGCAGATGGCCTACCGGTTCAACGGCATCG
GAGTGACCCAGAATGTGCTGTACGAGAACCAGAAGCTGATCGCCAACCAGTTCAACAGCGCC
ATCGGCAAGATCCAGGACAGCCTGAGCAGCACAGCAAGCGCCCTGGGAAAGCTGCAGGACGT
GGTCAACCAGAATGCCCAGGCACTGAACACCCTGGTCAAGCAGCTGTCCTCCAACTTCGGCG
CCATCAGCTCTGTGCTGAACGATATCCTGAGCAGACTGGACAAGGTGGAAGCCGAGGTGCAG
ATCGACAGACTGATCACCGGAAGGCTGCAGTCCCTGCAGACCTACGTTACCCAGCAGCTGAT
CAGAGCCGCCGAGATTAGAGCCTCTGCCAATCTGGCCGCCACCAAGATGTCTGAGTGTGTGC
TGGGCCAGAGCAAGAGAGTGGACTTTTGCGGCAAGGGCTACCACCTGATGAGCTTCCCTCAG
TCTGCCCCTCACGGCGTGGTGTTTCTGCACGTGACATACGTGCCCGCTCAAGAGAAGAATTT
CACCACCGCTCCAGCCATCTGCCACGACGGCAAAGCCCACTTTCCTAGAGAAGGCGTGTTCG
TGTCCAACGGCACCCATTGGTTCGTGACCCAGCGGAACTTCTACGAGCCCCAGATCATCACC
ACCGACAACACCTTCGTGTCTGGCAACTGCGACGTCGTGATCGGCATTGTGAACAATACCGT
GTACGACCCTCTGCAGCCCGAGCTGGACAGCTTCAAAGAGGAACTGGATAAGTACTTTAAGA
ACCACACAAGCCCCGACGTGGACCTGGGCGATATCAGCGGAATCAATGCCAGCGTCGTGAAC
ATCCAGAAAGAGATCGACCGGCTGAACGAGGTGGCCAAGAATCTGAACGAGAGCCTGATCGA
CCTGCAAGAACTGGGGAAGTACGAGCAGTACATCAAGTGGCCCTGGTACATCTGGCTGGGCT
TTATCGCCGGACTGATTGCCATCGTGATGGTCACAATCATGCTGTGTTGCATGACCAGCTGC
TGTAGCTGCCTGAAGGGCTGTTGTAGCTGTGGCAGCTGCTGCAAGTTCGACGAGGACGATTC
TGAGCCCGTGCTGAAGGGCGTGAAACTGCACTACACCTAA

In embodiments, the bacterial antigen comprises but is not limited to an antigen from a mycobacterium, Listeria, Chlamydia, Rickettsia, Yersinia, Helicobacter, Legionella, Streptococcus, Staphylococcus, Neisseria, or Salmonella.

In embodiments, the parasitic antigen comprises but not limited to an antigen from a plasmodium or trypanosome.

A further aspect of the invention relates to a pharmaceutical composition comprising a replication-deficient murine Cytomegalovirus (MCMV) vector suitable to induce an antigen-specific immune response in a subject, wherein said vector expresses a disease antigen, and wherein the composition is configured for administration to a non-mouse subject.

By way of example, a composition can be configured for administration in a non-mouse subject by determining appropriate administration forms, doses, volumes and formulations. A skilled person is capable of assessing any given composition form intended for administration and determining whether it is suitable or intended for administration in a mouse and/or other subject. For example, larger doses, larger volumes, formulation type e.g. tablet, injection solution, or other forms, formulation packaging and/or other formulation aspects will clearly indicate for example use in humans compared to use in a mouse.

A further aspect of the invention relates to a pharmaceutical composition comprising a replication-deficient murine Cytomegalovirus (MCMV) vector, wherein said vector has a disrupted immediate-early 2 (ie2) gene causing a replication deficiency of said vector in a non-mouse subject, wherein said vector expresses a disease antigen and wherein the composition is configured for administration to a non-murine subject.

Such a pharmaceutical composition is able to induce an antigen-specific immune response against e.g. an infectious disease or cancer in a host without spreading in the host. This increases the safety level of the composition for administration to a human or non-human animal. The pharmaceutical composition can be configured for administration as a vaccine. It is advantageous that such vaccine comprises a viral vector comprising a replication-deficient MCMV, which per se does not induce or induces to a low extent an unwanted immune response. This unwanted immune response limits the antigen delivery to cells upon repeat administration and facilitates the induction of recall immune response against viral vector antigens at the expense of immune priming.

Based on the surprising finding that the vaccine comprising the contemplated viral vector is replication deficient in the non-mouse subject, one advantage is that the contemplated viral vector will not spread in the host, nor elicit immune priming in the host raised by viral vector per se. Meanwhile, a robust immune protection can be obtained against a wide range of infectious diseases and cancers depending on selection of the disease antigen.

In embodiments of the invention, the vaccine according to the present invention is not only able to establish a life-long inflationary CD8+ T-cell response, but also induce a B-cell based humoral responses, which prevent viral spread via extracellular fluids. It has been demonstrated that neutralizing humoral immune responses against pandemic viruses, e.g. influenza virus, SARS-CoV-2 were induced by vaccination of contemplated vaccine. Humoral immunity elicited by contemplated vaccination provides better protection against pandemic virus, e.g. influenza virus, SARS-COV-2 the robust cellular immunity elicited by contemplated vaccination.

A further advantage of the contemplated vaccine is to provide long-term immunity. In embodiments, the levels of neutralizing antibodies may increase over time, such that the levels of class-switched isotypes gradually increase at later time points after vaccination, which may be concomitant with an increase in avidity. Without being bound by theory, the continuous restimulation of antigen-specific B cells by sparse antigen expression during latency, resulting from replication deficiency of the contemplated viral vector, represents a further potential benefit in an efficient vaccine.

In embodiments, the pharmaceutical composition is a vaccine, preferably configured for single-dose administration.

Single dose administration may be advantageous, for example in settings or regions where a lack or limitation of facilities for manufacturing the viral vector vaccine are evident, or in settings or regions with restricted cold-chain storage capacity. The single dose vaccination can help accelerate pandemic control, as vaccine coverage would be doubled using a single dose vaccine rather than a two or multi-dose regimen. A single dose vaccination could also help mitigate adverse events, such as fever, headaches, chills and fatigue, which have been reported at higher rates with a second dose or further dose of a vaccine.

Based on the findings shown herein, a long-term immune protection including neutralizing antibody responses may be obtained in a single vaccination dose, thus obviating the need for booster vaccinations.

In embodiments, the pharmaceutical composition further comprises a human or veterinary acceptable carrier, excipient, vehicle and/or adjuvant. Adjuvants and suitable formulations are known to a skilled person and may be selected accordingly in order to produce a composition suitable for administration in any given target subject. In embodiments, the pharmaceutical composition is for use in inducing an antigen-specific immune response in a non-mouse subject to the expressed disease antigen.

A further aspect relates to a method for inducing an antigen-specific immune response in a subject, comprising administering replication deficient murine Cytomegalovirus (MCMV) vector, wherein the subject is not a mouse, and wherein said vector expresses a disease antigen.

In embodiments, the vector has a disrupted immediate-early 2 (ie2) gene, causing a replication deficiency of said vector in a non-mouse, preferably human, subject.

In embodiments, an open reading frame for the disease antigen is inserted in the vector to replace the ie2 open reading frame or part thereof.

A further aspect relates to a method for inducing an antigen-specific immune response in a subject, comprising administering a pharmaceutical composition comprising a replication-deficient murine Cytomegalovirus (MCMV) vector, wherein said vector expresses a disease antigen, and wherein the composition is configured for administration to a non-mouse subject, preferably comprising a human or veterinary acceptable carrier, excipient, vehicle and/or adjuvant.

In embodiments, the vector has a disrupted immediate-early 2 (ie2) gene causing a replication deficiency of said vector in a non-mouse, preferably human, subject.

In embodiments, the pharmaceutical composition is a vaccine, preferably configured for single-dose administration.

The embodiments describing the medical use of the invention may be used to describe the pharmaceutical composition comprising the vector of the invention and a method for inducing an antigen-specific immune response in a subject, and vice versa. This also applies to any embodiments used to describe the pharmaceutical composition and a method for inducing an antigen-specific immune response in a subject. The invention is unified by the novel and beneficial replication-deficient murine Cytomegalovirus (MCMV) vector for use in inducing an antigen-specific immune response in a non-mouse subject and thus the relevant features described herein for one aspect may be used to describe any given aspect of the invention, in a manner in conformity with the understanding of a skilled person.

### DETAILED DESCRIPTION OF THE INVENTION

All cited documents of the patent and non-patent literature are hereby incorporated by reference in their entirety.

The present invention relates a replication-deficient murine cytomegalovirus vector for use in inducing an antigen-specific immune response in a subject, wherein the subject is not a mouse subject, and wherein said vector expresses a disease antigen. The invention further relates to a pharmaceutical composition comprising a replication-deficient murine cytomegalovirus vector, wherein said vector preferably has a disrupted immediate-early 2 (ie2) gene causing a replication deficiency of said vector in a non-murine subject, wherein said vector expresses a disease antigen and wherein the composition is configured for administration to a non-mouse subject. The invention further relates to a pharmaceutical composition for use in inducing an antigen-specific immune response in a non-mouse subject to the expressed disease antigen.

In embodiments, the subject is not a murine subject. In embodiments, the vector has a replication deficiency in a non-murine subject. In embodiments, the composition is configured for administration to a non-murine subject. In embodiments, any mention of “non-mouse” may also refer to “non-murine”.

The replication-deficient murine cytomegalovirus (MCMV) vector is employed to induce an antigen-specific immune response in a subject. The term “replication deficient” in the context of the MCMV vector contemplated herein shall mean replication-incompetent or completely unable to spread beyond the initial non-murine host cell, preferably beyond a mouse cell. Replication deficiency may also refer to an undetectable replication of the virus in the host.

In embodiments, the replication deficient MCMV vector used herein can establish latency and thereby provide repeated stimulation to the immune system but cannot produce infectious virus particles.

In embodiments, a replication-deficient MCMV comprises a gene disruption causing replication incompetency or deficiency. The contemplated disrupted gene can be one or more genes in MCMV targeting conserved cellular functions. The mutation of these genes leads to a replication solely in mouse cells and replication incompetency or deficiency in non-mouse subject. These genes include, but are not limited to viral genes targeting apoptotic molecules e.g. M36, M38.5, m41, those affecting promyelocytic leukemia (PML) associated nuclear bodies (e.g. IE1) or interferon signaling (e.g. M35, M27) or immediate-early 2 (ie2). The skilled person knows such genes in the art and can identify the replication deficiency resulting from the disruption of such genes according to art.

In one embodiment, the vector as used herein has a disrupted immediate-early 2 (ie2) gene, causing a replication deficiency of said vector in a non-mouse subject. In embodiments, the ie2 gene is disrupted by deletion of the ie2 gene or part thereof.

As used herein the immediate-early 2 (ie2), UniProtKB-P24909, refers to murine cytomegalovirus α (immediate-early) gene product which is a protein that is related to the human cytomegalovirus US22 protein family. This murine ie2 is dispensable for growth and latency of MCMV vector in mice, but is indispensable for growth in non-murine subject.

In embodiments, a disrupted ie2 causes a replication deficiency in human or other non-murine cells due to its replication incompetency or replication inability in these subjects. In other embodiments, the replication-deficient MCMV vector as used herein comprises multiple inactivated essential genes required for generation of infectious viral particles.

As used herein “a disrupted ie2 gene” refers to a dysfunctional ie2 gene which consequently encodes a functionally deficient IE2 protein or does not encode any dysfunctional or functional IE2 protein. The disruption of ie2 can be achieved by methods known in the art. In embodiments, the ie2 gene can be disrupted by direct insertion or deletion or substitution of one or more nucleotides in the open reading frame of the ie2 gene. In embodiments, the ie2 gene can be inactivated by means of inactivating the operably linked sequence of the ie2 gene, preferably a promoter driving the expression of the ie2 gene or other epigenetic modification of the ie2 gene.

“Operably linked” shall mean a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein-coding regions, in the same reading frame.

As used herein the term “deletion” shall mean the removal of a DNA sequence, the region on either side of the removed sequence being joined together.

In embodiments, the ie2 gene is disrupted by deletion of a TATA box or deletion of the ie2 open reading frame or part thereof.

As used herein, “open reading frame (ORF)” refers to a series of nucleotide triplets (codons) coding for amino acids without any internal termination codons. These sequences are usually translatable into a peptide. The term “part of ie2 open reading frame” shall mean any nucleotide or nucleotide sequence in the ie2 open reading frame whose deletion or modification leads to a disruption of the ie2 gene or encoding a dysfunctional IE2 protein or a gene silencing of ie2.

As used herein, “TATA box” shall mean a sequence of DNA located in the core promoter region of a gene. The TATA box is a non-coding DNA sequence and contains a consensus sequence characterized by repeating T and A base pairs. Based on the sequence and mechanism of TATA box initiation, mutations such as insertions, deletions, and point mutations to the consensus sequence can result in phenotypic changes. In embodiments, the deletion of a TATA box leads to disruption of ie2 gene or encoding a dysfunctional IE2 protein or a gene silencing of ie2.

As used herein, the term “virus” shall have its ordinary meaning in the art. A virus consists essentially of a core of a single nucleic acid surrounded by a protein coat (capsid) and has the ability to replicate only inside a living cell. “Viral replication” is the production of additional virus particles by the occurrence of at least one viral life cycle. A virus may subvert the host cells' normal functions, causing the cell to behave in a manner determined by the virus.

As used herein, “cytomegalovirus (CMV)” refers to a member of the beta subclass of the family of herpesviruses. CMV is a relatively large (containing a 230 kilobase genome), double stranded DNA virus, with host-range specific variants such as MCMV (murine CMV) and HCMV (human CMV).

The term “murine cytomegalovirus (MCMV)” refers to a herpesvirus of the subfamily betaherpesviridae. It is a double-stranded enveloped DNA virus with host specificity for mice. Murine cytomegalovirus (MCMV) was first described in 1954, and it has been used to model human cytomegalovirus (HCMV) diseases in mice. MCMV is a natural pathogen of mice that is present in wild mice populations. MCMV can be determined by sequence analysis. For example, the complete DNA sequence of the Smith strain of murine cytomegalovirus (MCMV) was determined from virion DNA by using a whole-genome shotgun approach (Rawlinson, J Virol 1996 December; 70 (12): 8833-49). The genome has an overall G+C content of 58.7%, consists of 230,278 bp, and is arranged as a single unique sequence with short (31-bp) terminal direct repeats and several short internal repeats. Sequence variation can occur and can be detected by a skilled person.

The term “vector” refers to any carrier of exogenous DNA or RNA that is useful for transferring exogenous DNA to a host cell, for example for replication and/or appropriate expression of the exogenous DNA by the host cell. Vector nucleic acid molecules of particular sequences can be incorporated into a vector that is then introduced into a host cell, thereby producing a transformed host cell. Vectors can be viral vectors, such as CMV vectors. Viral vectors may be constructed from wild type or attenuated virus, including replication deficient virus.

In embodiments, the replication deficient MCMV vector is used for carrying exogenous DNA to a non-murine host cell for expression of exogenous DNA by the host cell. In embodiments, the vector as described herein expresses a disease antigen, wherein the disease antigen is expressed in the host cell.

The term “antigen” shall mean a substance that can stimulate e.g. the production of antibodies or a T cell response in a mammal, including compositions that are injected or absorbed into a mammal. An antigen reacts with the products of specific humoral or cellular immunity, including those induced by heterologous immunogens. The term “antigen” includes all related antigenic epitopes.

As used herein, the term “disease antigen” is interchangeable with “disease causing antigen” or “disease associated antigen”. A disease antigen can be expressed coincidentally with a particular disease process, where antigen expression correlates with or predicts development of that disease. Disease antigens include, for example, an oncogene, which is associated with certain types of cancer which is associated with this type of cancer. A disease antigen can be an antigen recognized by T-cells or B-cells. Some disease antigens may also be tissue-specific. A tissue-specific antigen is expressed in a limited number of tissues.

In embodiments, the disease antigen is a viral antigen comprising an antigen from but not limited to coronavirus, influenza virus, herpes virus, polyoma virus, papilloma virus, orthomyxovirus, paramyxovirus, picornavirus, parvovirus, reovirus, rhabdovirus, togavirus, African swine fever, adenovirus, coxsackie, arenavirus, retrovirus, alfavirus, pestivirus, a flavivirus or an arbovirus. In embodiments the viral antigen is a Spike, Envelope, Membrane, Nucleo-protein of SARS-COV-2 or any part thereof. In embodiments, the viral antigen is a hemagglutinin or neuraminidase of the influenza virus.

In embodiments, the disease antigen is a bacterial antigen comprising an antigen from but not limited to a Mycobacterium, Listeria, Chlamydia, Rickettsia, Yersinia, Helicobacter, Legionella, Streptococcus, Staphylococcus, Neisseria, or Salmonella.

In embodiment, the disease antigen is a parasitic antigen comprising an antigen from but not limited to a plasmodium or trypanosome.

The term “any part thereof” in the context of disease antigen according to the present invention shall mean any fragment(s) thereof comprising more than one amino acid and being able to induce the immune response as the whole part of the antigen or correlates with the disease caused by the whole part of the disease antigen. It includes for example an epitope, also called an antigenic determinant of the disease antigen, which are particular chemical groups or peptide sequences on a molecule that are antigenic, such that it elicits a specific immune response. For example, an epitope may be a tumor-derived polypeptide that is expressed form a nucleic acid delivered to a cell by a recombinant-deficient MCMV vector, preferably in form of a vaccine.

The term “any part thereof” further refers to a portion of a polypeptide that exhibits at least one useful epitope. The phrase “any part thereof” therefore refers to all fragments of a polypeptide that retain an activity, or a measurable portion of an activity, of the polypeptide from which the fragment is derived. Fragments, for example, can vary in size from a polypeptide fragment as small as an epitope capable of binding an antibody molecule to a large polypeptide capable of participating in the characteristic induction or programming of phenotypic changes within a cell. An epitope is a region of a polypeptide capable of binding an immunoglobulin generated in response to contact with an antigen.

As used herein, the term “cancer” shall mean malignant neoplasm that has undergone characteristic anaplasia with loss of differentiation, increased rate of growth, invasion of surrounding tissue, and/or is capable of metastasis. As used herein, the term “tumor” is a neoplasm. This term includes solid and hematological tumors.

Examples of hematological tumors include, but are not limited to: leukemias, including acute leukemias (such as acute lymphocytic leukemia, acute myelocytic leukemia, acute myelogenous leukemia and myeloblastic, promyelocytic, myelomonocytic, monocytic and erythroleukemia), chronic leukemias (such as chronic myelogenous leukemia, and chronic lymphocytic leukemia), myelodysplastic syndrome, and myelodysplasia, polycythemia vera, lymphoma, (such as Hodgkin's disease, all forms of non-Hodgkin's lymphoma), multiple myeloma, Waldenstrom's macroglobulinemia, and heavy chain disease.

Examples of solid tumors, such as sarcomas and carcinomas, include, but are not limited to: fibrosarcoma, myxosarcoma, liposarcoma, chondrosarcoma, osteogenic sarcoma, and other sarcomas, synovioma, mesothelioma, Ewing's tumor, leiomyosarcoma, rhabdomyosarcoma, colon carcinoma, pancreatic cancer, breast cancer, lung cancer, ovarian cancer, prostate cancer, hepatocellular carcinoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, bladder carcinoma, melanoma, and CNS tumors (such as a glioma, astrocytoma, medulloblastoma, craniopharyogioma, ependymoma, pinealoma, hemangioblastoma, acoustic neuroma, oligodendroglioma, menangioma, meningioma, neuroblastoma and retinoblastoma).

As used herein, the term “antigen of cancer cell” shall mean a tumor antigen which can stimulate tumor-specific T cell-defined immune responses or antibodies to tumor cells. An immunogenic composition, such as a cancer vaccine, can include one or more tumor antigen.

In embodiments, the disease antigen is expressed in the vector as described herein. In this context, the disease antigen can be placed at any location of the vector where the disease antigen can be under control of operably linked sequence, preferably a promoter driving the expression of the disease antigen. In some embodiment, the operably linked sequence is a promoter including an inducible promoter. In embodiment, a reading frame for the disease antigen driven by an immediate-early MCMV promoter is inserted in the vector to replace any non-essential viral genomic sequences.

The term “non-essential viral genomic sequence” shall mean any viral gene that is not required for virus replication in cells, preferably in murine cells, more preferably in mouse cells, for example, M34, ie1, ie2 or ie3. Non-essential genes in cells may play a role in viral pathogenesis in the host. There are a number of techniques used to identify essential and non-essential viral genes, already known in the art. For example, RNA interference or inhibition (RNAi) has been used to identify essential and non-essential ranavirus genes (Sample et al., 2007, Virology. 2007 February 20; 358 (2): 311-20; Whitley et al., 2011, Dev Comp Immunol. 2011 September; 35 (9): 937-48, Xie 2004 et al., Virus Res. 2014 Aug. 30; 189 ( ): 214-25). This approach, by way of example, is where ORFs are inhibited, or knocked down, from being expressed at the mRNA level, and viral growth can be used to measure the essentialness of the target ORF.

In embodiments, the disease antigen is inserted in the vector to replace the ie2 open reading frame or part thereof. In embodiments, a replication deficient MCMV vector as described herein can be generated by inserting the gene of the disease antigen in place of the ie2 gene by replacing the start and stop codon of the ie2 ORF with the start and stop codon of the open reading frame of the disease antigen. For example, the replication-deficient MCMV vector expressing SARS-COV-2 spike can be generated by inserting the full-length spike ORF with the start and stop codon of the spike ORF.

In embodiments, the disease antigen is a spike protein of a coronavirus, preferably of SARS-CoV-2.

Coronaviruses are a group of related viruses that cause diseases in mammals and birds. The scientific name for coronavirus is Orthocoronavirinae or Coronavirinae. Coronavirus belongs to the family of Coronaviridae. The family is divided into Coronavirinae and Torovirinae sub-families, which are further divided into six genera: Alphacoronavirus, Betacoronavirus, Gammacoronavirus, Deltacoronavirus, Torovirus, and Bafinivirus. While viruses in the genera Alphacoronaviruses and Betacoronaviruses infect mostly mammals, the Gammacoronavirus infect avian species and members of the Deltacoronavirus genus have been found in both mammalian and avian hosts.

In humans, coronaviruses cause respiratory tract infections that can be mild, such as some cases of the common cold, and others that can be lethal, such as SARS, MERS, and COVID-19. Coronaviruses are enveloped viruses with a positive-sense single-stranded RNA genome and a nucleocapsid of helical symmetry. The genome size of coronaviruses ranges from approximately 27 to 34 kilobases, the largest among known RNA viruses.

Various species of human coronaviruses are known, such as, without limitation, Human coronavirus OC43 (HCoV-OC43), of the genus β-CoV, Human coronavirus HKU1 (HCoV-HKU1), of the genus β-CoV, Human coronavirus 229E (HCoV-229E), α-CoV, Human coronavirus NL63 (HCoV-NL63), α-CoV, Middle East respiratory syndrome-related coronavirus (MERS-COV), severe acute respiratory syndrome coronavirus (SARS-COV), Severe acute respiratory syndrome coronavirus 2 (SARS-COV-2).

Coronaviruses vary significantly in risk factor. Some can kill more than 30% of those infected (such as MERS-COV), and some are relatively harmless, such as the common cold. Coronaviruses cause colds with major symptoms, such as fever, and a sore throat, e.g. from swollen adenoids, occurring primarily in the winter and early spring seasons. Coronaviruses can cause pneumonia (either direct viral pneumonia or secondary bacterial pneumonia) and bronchitis (either direct viral bronchitis or secondary bacterial bronchitis). Coronaviruses can also cause SARS.

Advances in nucleic acid sequencing technology (commonly termed Next-Generation Sequencing, NGS) are providing large sets of sequence data obtained from a variety of biological samples and allowing the characterization of both known and novel virus strains. Established methods are therefore available for determining a Coronavirus infection.

As used herein, the term “encode” shall mean a polynucleotide is said to encode a polypeptide if, in its native state or when manipulated by methods well known to those skilled in the art, it can be transcribed and/or translated to produce the mRNA for and/or the polypeptide or a fragment thereof.

As used herein, the term “expression” shall mean translation of a nucleic acid into a protein, for example the translation of a mRNA encoding a tumor antigen into a protein.

In embodiments, the immune response comprises a neutralizing antibody response or a T-cell response against the disease antigen. As used herein, the term “immune response” shall mean a change in immunity, for example a response of a cell of the immune system, such as a B-cell, T cell, macrophage, monocyte, or polymorphonucleocyte, to an immunogenic agent in a subject. The response can be specific for a particular antigen (an “antigen-specific response”). In some embodiments, an immune response is a T cell response, such as a CD4+ response or a CD8+ response. In another example, the response is a B-cell response, and results in the production of specific antibodies to the immunogenic agent. Immunity means the state of being able to mount a protective response upon exposure to an immunogenic agent. Protective responses can be antibody-mediated or immune cell-mediated, and can be directed toward a particular pathogen or tumor antigen. Immunity can be acquired actively (such as by exposure to an immunogenic agent, either naturally or in a pharmaceutical composition) or passively (such as by administration of antibodies or in vitro stimulated and expanded T cells).

In embodiments, immune response comprises a long-term immune protection preferably by neutralising antibody responses. Without being bound by theory, the long-term immunity may include continuous restimulation of antigen-specific B cells by sparse antigen expression during latency, which boosts B-cell immunity over time.

In some embodiments, an immune response provides protection for the subject from the immunogenic agent or the source of the immunogenic agent. For example, the response can treat a subject having a tumor, for example by interfering with the metastasis of the tumor or reducing the number or size of a tumor. An immune response can be active and involve stimulation of the subject's immune system, or be a response that results from passively acquired immunity. A “repeatedly stimulated” immune response is a long-term immune response resulting from the periodic and repetitive stimulation of the immune system by the repeated production of an antigen within a host.

In a particular embodiment, an increased or enhanced immune response is an increase in the ability of a subject to fight off a disease, such as a tumor, a viral infection, a bacterial infection or a parasitic infection.

As used herein, the term “neutralizing antibody” shall mean an antibody defending a target cell from a pathogen or infectious particle by neutralizing any effect it has biologically, preferably leading to stopping or inhibiting a pathogen infecting a target cell. Neutralizing a pathogen makes the pathogen no longer infectious or pathogenic. Neutralizing antibodies are part of the humoral response of the acquired immune system against viruses, intracellular bacteria and microbial toxins. In a particular embodiment, by binding specifically to Spike protein of SARS-COV-2, neutralizing antibodies prevent the virus particle from interacting with its ACE2 receptor of host cell it might infect and destroy.

In embodiments, the immune response comprises priming, maintaining and/or boosting the immune system of the subject. In embodiments, the priming, maintaining and/or boosting the immune system can be achieved by administrating two, preferably single doses of contemplated viral vectors. In embodiments, the priming, maintaining and/or boosting the immune system can be achieved by a T-cell response, preferably a CD8+ T-cell response. In embodiments, the priming, maintaining and/or boosting the immune system is achieved by a T-cell response accompanied by neuralising antibody response, preferably a long-term neuralising antibody response.

As used herein, the term “priming the immune system” shall mean a first contact of the antigenic specific T helper cell precursors and an antigen. It is essential to the T helper cells for the subsequent interaction with B cells to produce antibodies. Priming of antigen specific naïve lymphocytes occurs when antigen is presented to them in immunogenic form capable inducing an immune response. Subsequently, the primed cells will differentiate either into effector cells or into memory cells that can mount stronger and faster response to second and upcoming immune challenges.

The term antigen-specific T cell shall mean a CD8+ or CD4+ lymphocyte that recognizes a particular antigen. Generally, antigen-specific T cells specifically bind to a particular antigen presented by major histocompatibility complex (MHC) molecules, but not other antigens presented by the same MHC.

As used herein, the term “maintaining the immune system of the subject” shall mean one or more further contacts of the immune system with same antigen or fragment(s) thereof from the first contact while priming the immune system against an antigen or fragment(s) thereof. Alternative, the immune system can be maintained by a further contact with another antigen or fragment(s) thereof causing the same disease as the antigen from the first contact with the immune system. “Maintaining immune system” can be determined for example with an in vitro titration assay of neutralizing antibody or with animal model or any methods known in the art without undue burden.

As used herein, the term “boosting the immune system” shall mean improved the immune system against the disease antigen. In one embodiment, it means to maintain the immune system of the subject against the disease antigen for longer after the immunity developed from the first contact of an antigen starts to wane. In other embodiments, it means a retreat the immune system with the same antigen as same antigen or fragment(s) thereof from the priming of the immune system against an antigen or fragment(s) thereof. Alternative, the immune system can be boosted by a further contact with another antigen or fragment(s) thereof causing the same disease as the antigen from the priming of the immune system or maintaining of the immune system.

Maintaining and boosting immune system can also be provided after the subject had the disease caused by disease antigen.

As used herein, the term “subject” shall mean living multi-cellular vertebrate organisms, a category that includes both human and non-human mammals. This term encompasses both known and unknown individuals, such that there is no requirement that a person working with a sample from a subject know who the subject is, or even from where the sample was acquired.

“Mouse subject” is interchangeable with “murine subject” shall mean part of subfamily Murinae in the family of Muridae. In embodiments, the murine subject shall exclude rat or exclude any murine subject in which an ie2-deficient murine cytomegalovirus vector can spread or replicate. In embodiments, the murine subject is Mus musculus. A “non-mouse subject” is a subject excluding a mouse subject.

A further aspect of the invention relates to a pharmaceutical composition comprising a replication-deficient murine Cytomegalovirus (MCMV) vector, wherein said vector preferably has a disrupted immediate-early 2 (ie2) gene causing a replication deficiency of said vector in a non-murine subject, wherein said vector expresses a disease antigen and wherein the composition is configured for administration to a non-murine subject.

As used herein, the term “administration” shall mean administering an active compound or composition by any route known to one of skill in the art. Administration can be local or systemic. Examples of local administration include, but are not limited to, topical administration, subcutaneous administration, intramuscular administration, intrathecal administration, intrapericardial administration, intra-ocular administration, topical ophthalmic administration, or administration to the nasal mucosa or lungs by inhalational administration. Systemic administration includes but not limited to intravenous and intraperitoneal routes, for example by directing intravascular administration to the arterial supply for a particular organ.

In embodiment, the pharmaceutical composition as described herein is configured for administration to a non-murine subject. The pharmaceutical composition is in a form configured or optimized for any route of administration in an effective amount sufficient to induce an antigen-specific immune response in a non-murine subject. In embodiments, the pharmaceutical composition is a vaccine, preferably configured for single-dose administration.

On example of “configured for administration to a non-mouse or non-murine subject” relates to an injection unit for human use, as commonly used in the vaccine products of SARS-COV-2. A skilled person is capable of identifying a composition configured for human use, compared to a composition configured for mouse use. In embodiments, common injection preparations, or vials for preparing syringes for human injection, may be considered as compositions configured for non-murine use.

“Effective amount” shall mean a quantity sufficient to achieve a desired effect in a subject being treated. An effective amount of a composition, such as a vaccine, can be administered in a single dose, or in several doses, during a course of treatment. However, the effective amount of the compound will be dependent on the compound applied, the subject being treated, the severity and type of the affliction, and the manner of administration of the compound.

In an embodiment, a “single-dose” means an effective amount of composition for inducing an immune response to the disease antigen, wherein the immune response can be priming, maintaining or boosting the immune response. In a preferred embodiment, a single-dose of the vaccine comprising the ie2-deficient murine cytomegalovirus vector shall be able to prime, maintain and boost the immune system due to its replication deficiency and latency in the host. This constitutes a periodic and repetitive stimulation of the immune system by the repeated production of an antigen within a host.

As used herein, the term “vaccine” is an immunogenic composition that can be administered to a mammal, such as a human, to confer immunity, such as active immunity, to a disease or other pathological condition. Vaccines can be used prophylactically or therapeutically. Thus, vaccines can be used reduce the likelihood of developing a disease (such as a tumor or pathological infection) or to reduce the severity of symptoms of a disease or condition, limit the progression of the disease or condition (such as a tumor or a pathological infection), or limit the recurrence of a disease or condition (such as a tumor). In particular embodiments, a vaccine is a replication-deficient CMV expressing a heterologous antigen, such as a tumor associated antigen derived from a tumor of the lung, prostate, ovary, breast, colon, cervix, liver, kidney, bone, or a melanoma.

Pharmaceutical compositions for use in accordance with the present invention may be formulated in a conventional manner using one or more physiologically acceptable carriers including excipients, vehicle and/or adjuvant that facilitate processing the active agent into preparations that can be used pharmaceutically. Proper formulation is dependent upon the target subject, for example human or non-human animal and route of administration chosen.

Pharmaceutically acceptable carriers, excipients, vehicles or adjuvants as used herein are nontoxic to the cell or mammal being exposed thereto at the dosages and concentrations employed. Often the physiologically acceptable carrier is an aqueous pH buffered solution. Examples of physiologically acceptable carriers include, but not limited to, buffers such as phosphate, citrate, and other organic acids; antioxidants including, but not limited to, ascorbic acid; low molecular weight (less than about 10 residues) polypeptide; proteins, such as, but not limited to, serum albumin, gelatin, or immunoglobulins; hydrophilic polymers such as, but not limited to, polyvinylpyrrolidone; amino acids such as, but not limited to, glycine, glutamine, asparagine, arginine or lysine; monosaccharides, disaccharides, and other carbohydrates including, but not limited to, glucose, mannose, or dextrins; chelating agents such as, but not limited to, EDTA; sugar alcohols such as, but not limited to, mannitol or sorbitol; salt-forming counterions such as, but not limited to, sodium; and/or nonionic surfactants such as, but not limited to, TWEEN; polyethylene glycol (PEG), and PLURONICS.

The phraseology and terminology used herein is for the purpose of description and should not be regarded as limiting. The use of “including,” “comprising,” or “having,” “containing,” “involving,” and variations thereof herein, is meant to encompass the items listed thereafter and equivalents,

Unless otherwise noted, technical terms are used according to conventional usage. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

SEQUENCE LISTING:

| SEQ ID NO: | Sequence | |
|---|---|---|
| 1 | ccgtgctgattcatatgccatatgagtgtattagggggcttt | ie2 gene |
| | ccgcttgggaaattgggtaaaaagtccccgtattactcacat | |
| | agggggcgtttggctttgcaaattaggggatttcagtgcatt | |
| | tggcattaaaaactattggttctagtcataaaacgggcggag | |
| | ttAACCATATAAAAGCTGTCCCCCATGCCATTCGAGCCCAGA | |
| | GCAGGACGGACCGCGGCTCGATACGACCCTATCTACGTTaac | |
| | gagcgggtgaacggagccccccaggccgggtaaggttgcatt | |
| | gggctcggctatgggacggggatttggcccggtactttaata | |
| | tgggtggggtct | |
| 2 | ATGTTCGTGTTTCTGGTGCTGCTGCCTCTGGTGTCCAGCCAG | Nucleic |
| | TGTGTGAACCTGACCACAAGAACCCAGCTGCCTCCAGCCTAC | sequence |
| | ACCAACAGCTTTACCAGAGGCGTGTACTACCCCGACAAGGTG | encoding a |
| | TTCAGATCCAGCGTGCTGCACTCTACCCAGGACCTGTTCCTG | spike |
| | CCTTTCTTCAGCAACGTGACCTGGTTCCACGCCATCCACGTG | protein of |
| | TCCGGCACCAATGGCACCAAGAGATTCGACAACCCCGTGCTG | the SARS- |
| | CCCTTCAACGACGGGGTGTACTTTGCCAGCACCGAGAAGTCC | CoV-2 |
| | AACATCATCAGAGGCTGGATCTTCGGCACCACACTGGACAGC | |
| | AAGACCCAGAGCCTGCTGATCGTGAACAACGCCACCAACGTG | |
| | GTCATCAAAGTGTGCGAGTTCCAGTTCTGCAACGACCCCTTC | |
| | CTGGGCGTCTACTATCACAAGAACAACAAGAGCTGGATGGAA | |
| | AGCGAGTTCCGGGTGTACAGCAGCGCCAACAACTGCACCTTC | |
| | GAGTACGTGTCCCAGCCTTTCCTGATGGACCTGGAAGGCAAG | |
| | CAGGGCAACTTCAAGAACCTGCGCGAGTTCGTGTTCAAGAAC | |
| | ATCGACGGCTACTTCAAGATCTACAGCAAGCACACCCCTATC | |
| | AACCTCGTGCGGGATCTGCCTCAGGGCTTCTCTGCTCTGGAA | |
| | CCCCTGGTGGATCTGCCCATCGGCATCAACATCACCCGGTTT | |
| | CAGACACTGCTGGCCCTGCACAGAAGCTACCTGACACCTGGC | |
| | GATAGCAGCAGCGGATGGACAGCTGGTGCCGCCGCTTACTAT | |
| | GTGGGCTACCTGCAGCCTAGAACCTTCCTGCTGAAGTACAAC | |
| | GAGAACGGCACCATCACCGACGCCGTGGATTGTGCCCTTGAT | |
| | CCTCTGAGCGAGACAAAGTGCACCCTGAAGTCCTTCACCGTG | |
| | GAAAAGGGCATCTACCAGACCAGCAACTTCCGGGTGCAGCCC | |
| | ACCGAATCCATCGTGCGGTTCCCCAATATCACCAATCTGTGC | |
| | CCCTTCGGCGAGGTGTTCAATGCCACCAGATTCGCCTCTGTG | |
| | TACGCCTGGAACCGGAAGCGGATCAGCAATTGCGTGGCCGAC | |
| | TACTCCGTGCTGTACAACTCCGCCAGCTTCAGCACCTTCAAG | |
| | TGCTACGGCGTGTCCCCTACCAAGCTGAACGACCTGTGCTTC | |
| | ACAAACGTGTACGCCGACAGCTTCGTGATCCGGGGAGATGAA | |
| | GTGCGGCAGATTGCCCCTGGACAGACAGGCAAGATCGCCGAC | |
| | TACAACTACAAGCTGCCCGACGACTTCACCGGCTGTGTGATT | |
| | GCCTGGAACAGCAACAACCTGGACTCCAAAGTCGGCGGCAAC | |
| | TACAATTACCTGTACCGGCTGTTCCGGAAGTCCAATCTGAAG | |
| | CCCTTCGAGCGGGACATCTCCACCGAGATCTATCAGGCCGGC | |
| | AGCACCCCTTGTAACGGCGTGGAAGGCTTCAACTGCTACTTC | |
| | CCACTGCAGTCCTACGGCTTTCAGCCCACAAATGGCGTGGGC | |
| | TATCAGCCCTACAGAGTGGTGGTGCTGAGCTTCGAACTGCTG | |
| | CATGCCCCTGCCACAGTGTGCGGCCCTAAGAAAAGCACCAAT | |
| | CTCGTGAAGAACAAATGCGTGAACTTCAACTTCAACGGCCTG | |
| | ACCGGCACCGGCGTGCTGACAGAGAGCAACAAGAAGTTCCTG | |
| | CCATTCCAGCAGTTTGGCCGGGATATCGCCGATACCACAGAC | |
| | GCCGTTAGAGATCCCCAGACACTGGAAATCCTGGACATCACC | |
| | CCTTGCAGCTTCGGCGGAGTGTCTGTGATCACCCCTGGCACC | |
| | AACACCAGCAATCAGGTGGCAGTGCTGTACCAGGACGTGAAC | |
| | TGTACCGAAGTGCCCGTGGCCATTCACGCCGATCAGCTGACA | |
| | CCTACATGGCGGGTGTACTCCACCGGCAGCAATGTGTTTCAG | |
| | ACCAGAGCCGGCTGTCTGATCGGAGCCGAGCACGTGAACAAT | |
| | AGCTACGAGTGCGACATCCCCATCGGCGCTGGCATCTGTGCC | |
| | AGCTACCAGACACAGACAAACAGCCCCAGACGGGCCAGATCT | |
| | GTGGCCAGCCAGAGCATCATTGCCTACACAATGTCTCTGGGC | |
| | GCCGAGAACAGCGTGGCCTACTCCAACAACTCTATCGCTATC | |
| | CCCACCAACTTCACCATCAGCGTGACCACAGAGATCCTGCCT | |
| | GTGTCCATGACCAAGACCAGCGTGGACTGCACCATGTACATC | |
| | TGCGGCGATTCCACCGAGTGCTCCAACCTGCTGCTGCAGTAC | |
| | GGCAGCTTCTGCACCCAGCTGAATAGAGCCCTGACAGGGATC | |
| | GCCGTGGAACAGGACAAGAACACCCAAGAGGTGTTCGCCCAA | |
| | GTGAAGCAGATCTACAAGACCCCTCCTATCAAGGACTTCGGC | |
| | GGCTTCAATTTCAGCCAGATTCTGCCCGATCCTAGCAAGCCC | |
| | AGCAAGCGGAGCTTCATCGAGGACCTGCTGTTCAACAAAGTG | |
| | ACACTGGCCGACGCCGGCTTCATCAAGCAGTATGGCGATTGT | |
| | CTGGGCGACATTGCCGCCAGGGATCTGATTTGCGCCCAGAAG | |
| | TTTAACGGACTGACAGTGCTGCCACCACTGCTGACCGATGAG | |
| | ATGATCGCCCAGTACACATCTGCCCTGCTGGCCGGCACAATC | |
| | ACAAGCGGCTGGACATTTGGAGCTGGCGCCGCTCTGCAGATC | |
| | CCCTTTGCTATGCAGATGGCCTACCGGTTCAACGGCATCGGA | |
| | GTGACCCAGAATGTGCTGTACGAGAACCAGAAGCTGATCGCC | |
| | AACCAGTTCAACAGCGCCATCGGCAAGATCCAGGACAGCCTG | |
| | AGCAGCACAGCAAGCGCCCTGGGAAAGCTGCAGGACGTGGTC | |
| | AACCAGAATGCCCAGGCACTGAACACCCTGGTCAAGCAGCTG | |
| | TCCTCCAACTTCGGCGCCATCAGCTCTGTGCTGAACGATATC | |
| | CTGAGCAGACTGGACAAGGTGGAAGCCGAGGTGCAGATCGAC | |
| | AGACTGATCACCGGAAGGCTGCAGTCCCTGCAGACCTACGTT | |
| | ACCCAGCAGCTGATCAGAGCCGCCGAGATTAGAGCCTCTGCC | |
| | AATCTGGCCGCCACCAAGATGTCTGAGTGTGTGCTGGGCCAG | |
| | AGCAAGAGAGTGGACTTTTGCGGCAAGGGCTACCACCTGATG | |
| | AGCTTCCCTCAGTCTGCCCCTCACGGCGTGGTGTTTCTGCAC | |
| | GTGACATACGTGCCCGCTCAAGAGAAGAATTTCACCACCGCT | |
| | CCAGCCATCTGCCACGACGGCAAAGCCCACTTTCCTAGAGAA | |
| | GGCGTGTTCGTGTCCAACGGCACCCATTGGTTCGTGACCCAG | |
| | CGGAACTTCTACGAGCCCCAGATCATCACCACCGACAACACC | |
| | TTCGTGTCTGGCAACTGCGACGTCGTGATCGGCATTGTGAAC | |
| | AATACCGTGTACGACCCTCTGCAGCCCGAGCTGGACAGCTTC | |
| | AAAGAGGAACTGGATAAGTACTTTAAGAACCACACAAGCCCC | |
| | GACGTGGACCTGGGCGATATCAGCGGAATCAATGCCAGCGTC | |
| | GTGAACATCCAGAAAGAGATCGACCGGCTGAACGAGGTGGCC | |
| | AAGAATCTGAACGAGAGCCTGATCGACCTGCAAGAACTGGGG | |
| | AAGTACGAGCAGTACATCAAGTGGCCCTGGTACATCTGGCTG | |
| | GGCTTTATCGCCGGACTGATTGCCATCGTGATGGTCACAATC | |
| | ATGCTGTGTTGCATGACCAGCTGCTGTAGCTGCCTGAAGGGC | |
| | TGTTGTAGCTGTGGCAGCTGCTGCAAGTTCGACGAGGACGAT | |
| | TCTGAGCCCGTGCTGAAGGGCGTGAAACTGCACTACACCTAA | |
| 3 | AACCATATAAAAGCTGTCCCCCATGCCATTCGAGCCCAGAGC | Deleted |
| | AGGACGGACCGCGGCTCGATACGACCCTATCTACGTT | Sequence |
| | | in IE2 |
| 4 | GCCCAGAGCAGGACGGACCGCGGCTCGATACGACCCTATCTA | Exon 1 of |
| | CGTT | ie2 gene |

### FIGURES

The following figures are presented to describe particular embodiments of the invention, without being limiting in scope.

### BRIEF DESCRIPTION OF THE FIGURES

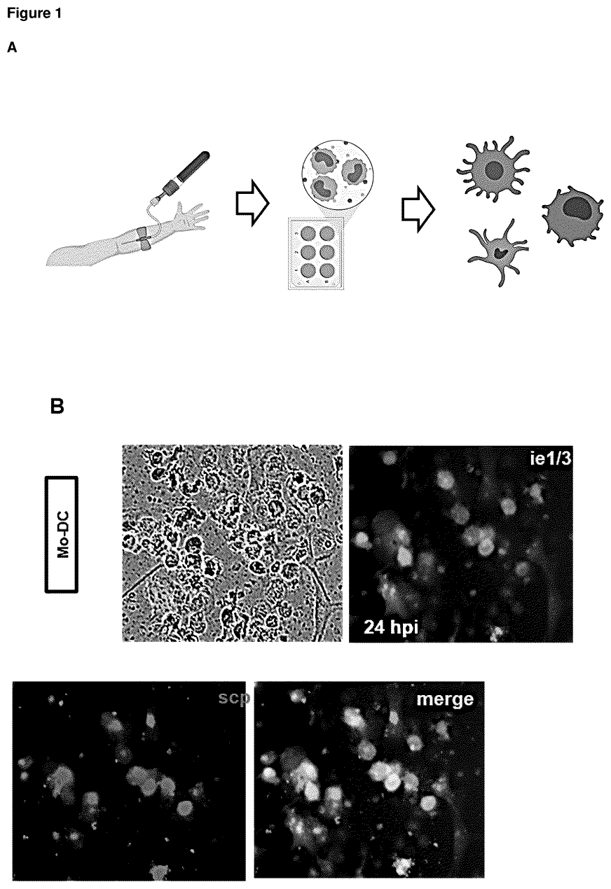

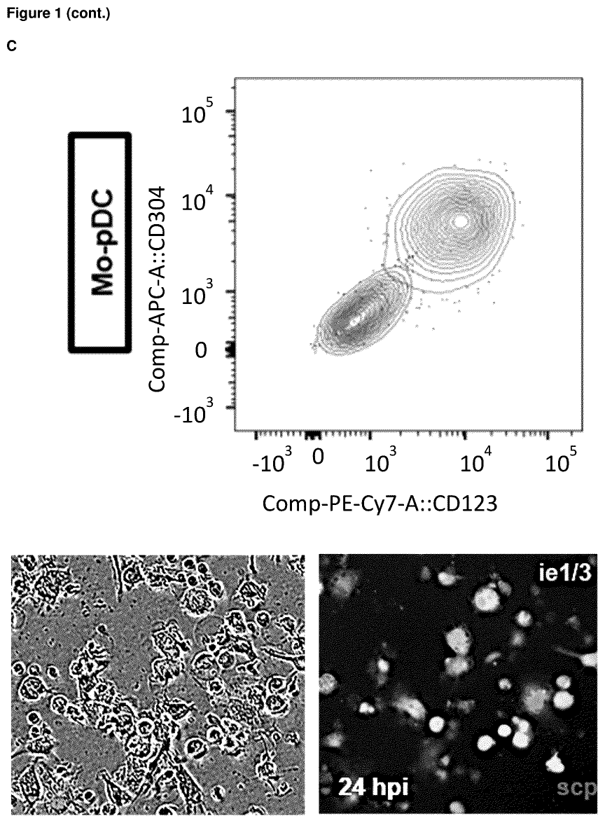

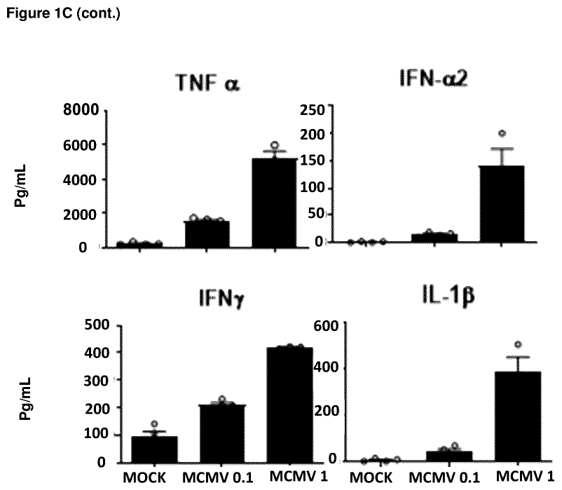

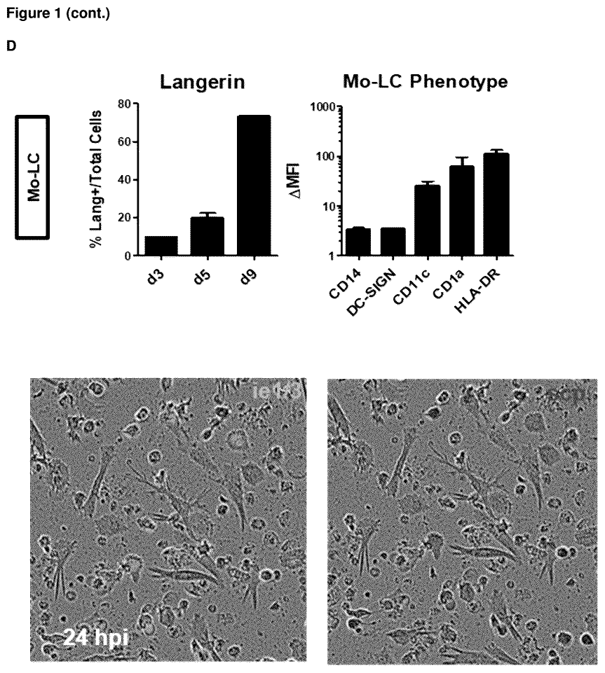

FIG. 1: Different kinds of functional antigen presenting cells induced from human monocyte by different cytokines are infected with MCMV.

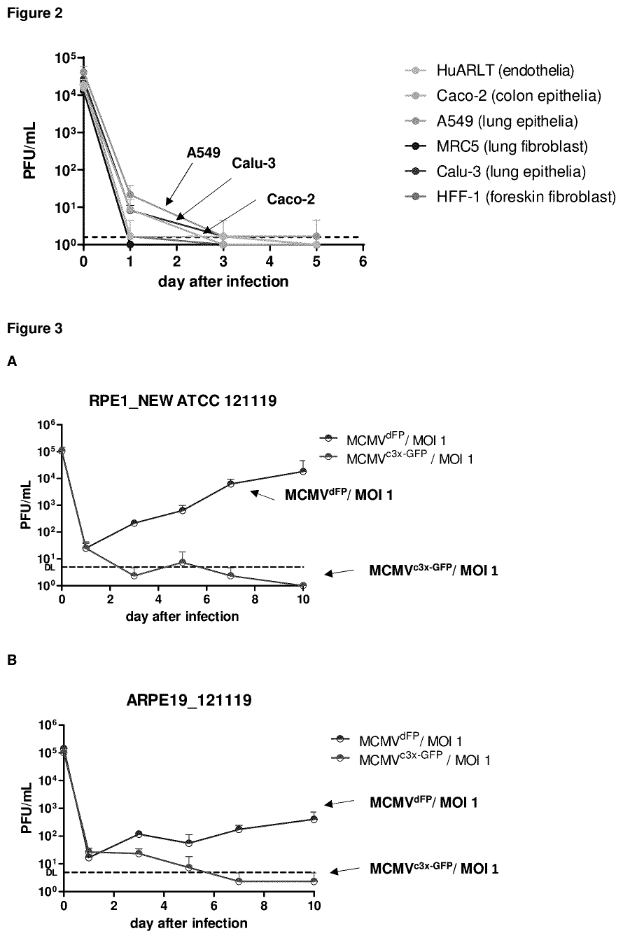

FIG. 2: MCMV ability to replicate in human cells.

FIG. 3: Retinal pigment epithelial cells are permissive to MCMVs.

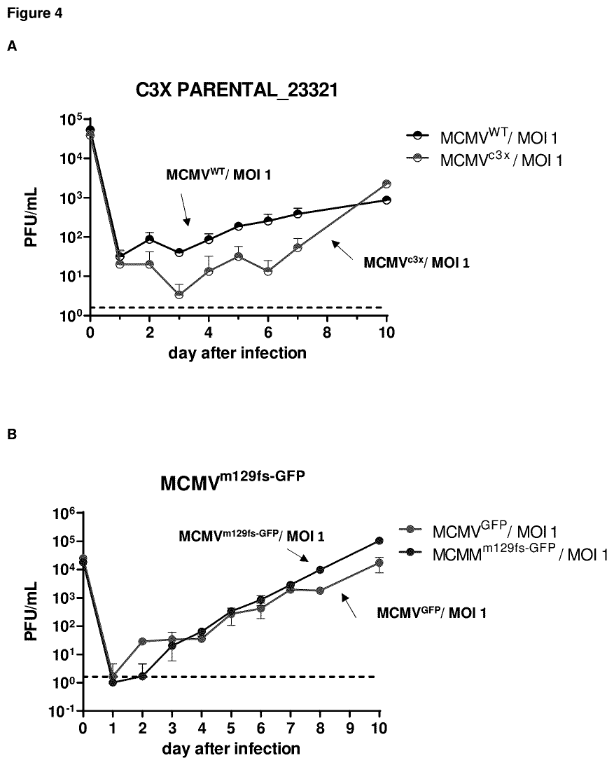

FIGS. 4: M129 and m131 mutants do not explain RPE growth phenomenon. Hence, these genes are not required for growth in RPE cells.

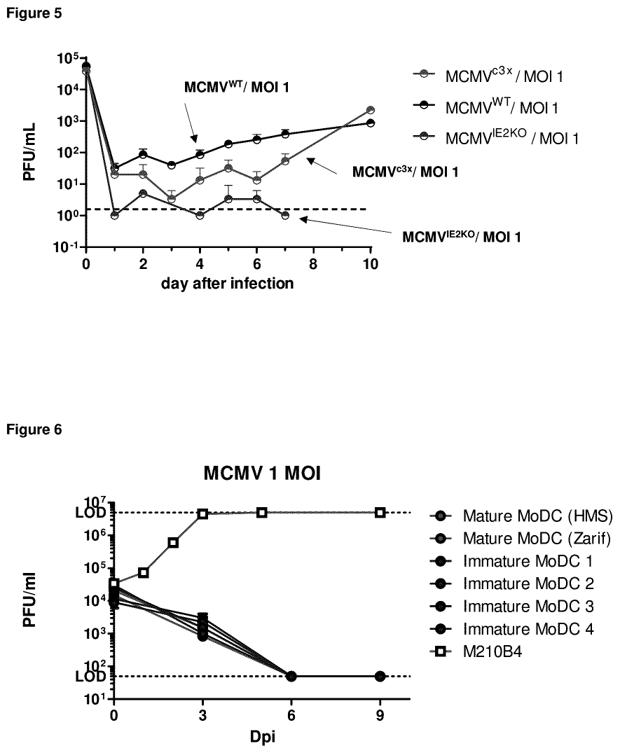

FIG. 5: The growth potential of RPE cells infected different MCMV mutants.

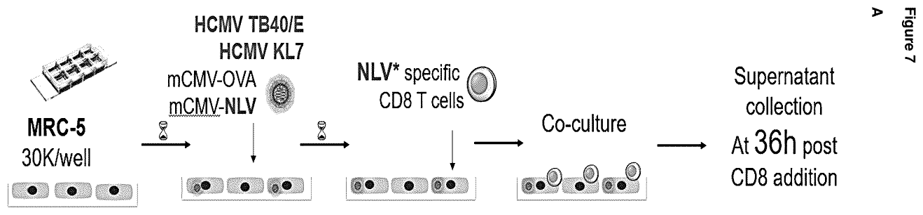

FIG. 6: MCMV does not proliferate in MoDC.

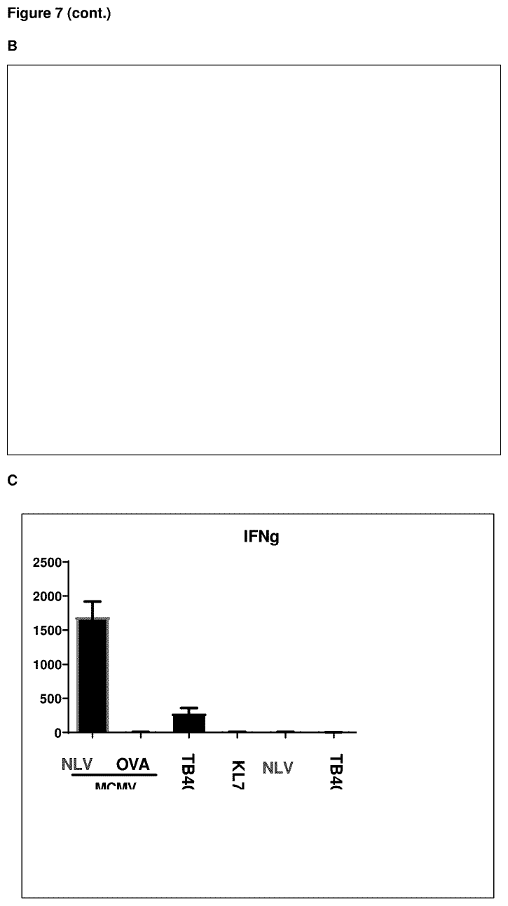

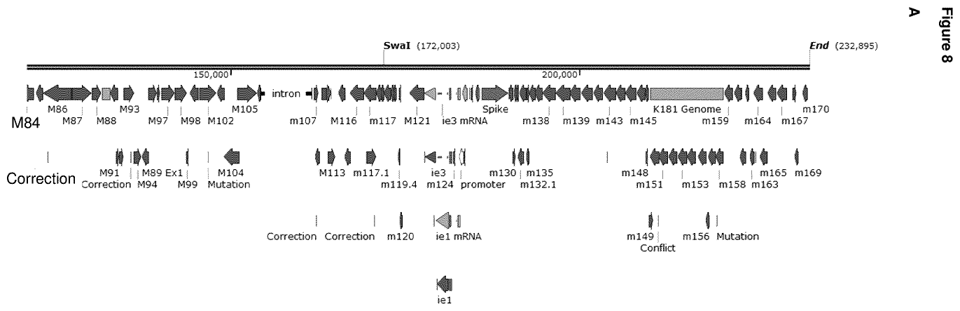

FIG. 7: Antigen-specific CD8 responses are stronger to cells infected with MCMV than HCMV.

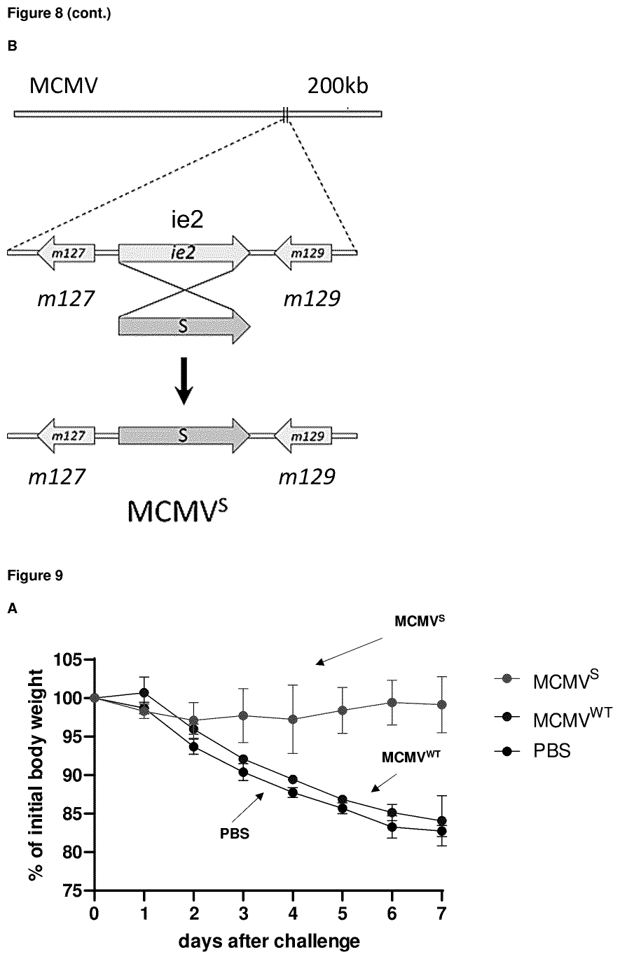

FIG. 8: Schematic images of the recombinant MCMV vector genome.

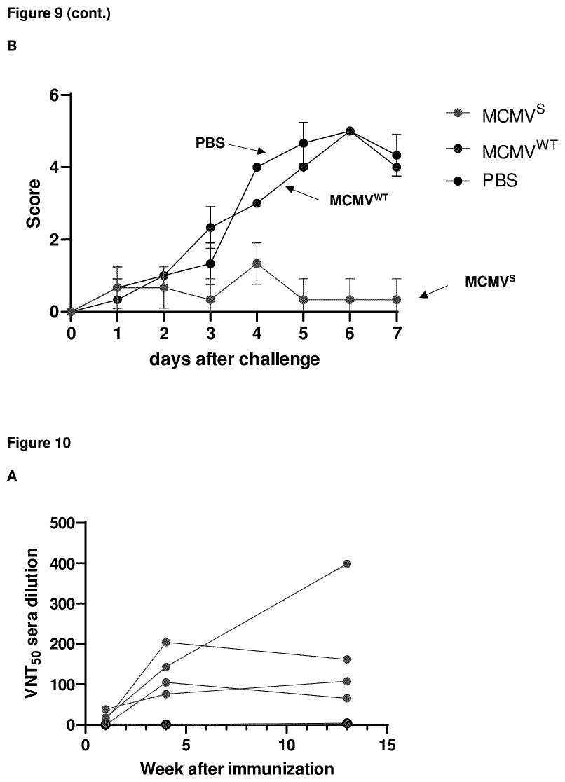

FIG. 9: MCMV immunized hamster weight loss and score upon SARS-COV-2 challenge.

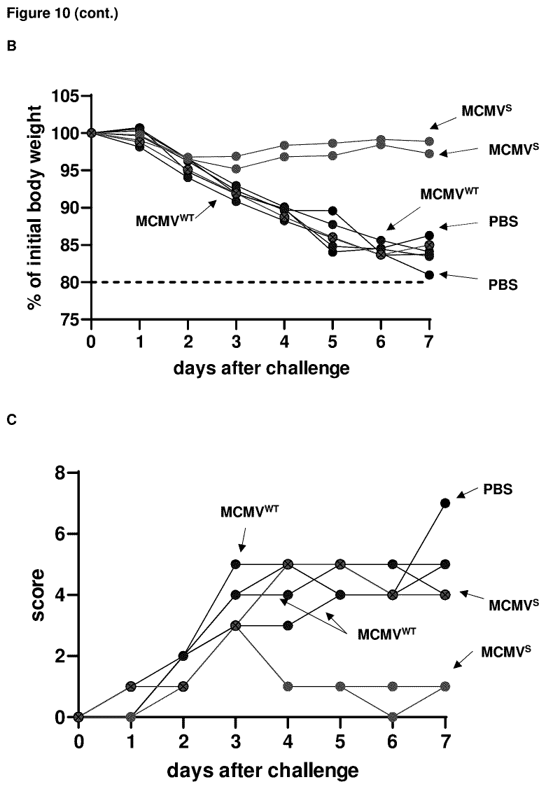

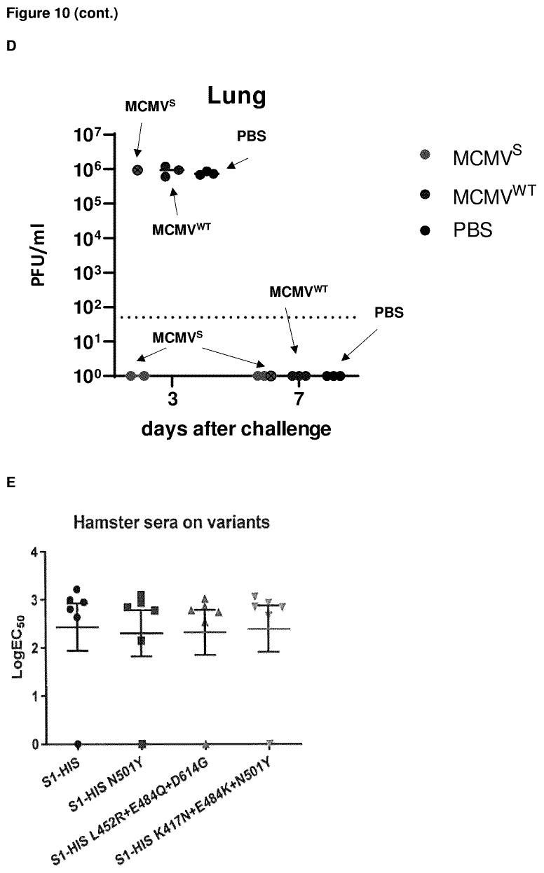

FIG. 10: MCMV immunized hamster weight loss and score upon SARS-COV-2 challenge.

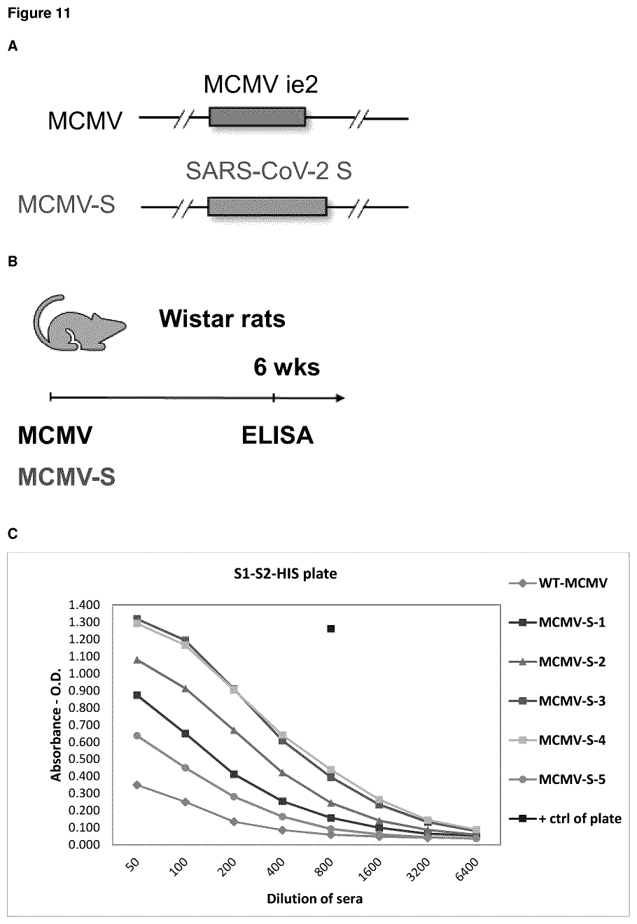

FIG. 11: Infection with MCMV-S induces antibody response in rats.

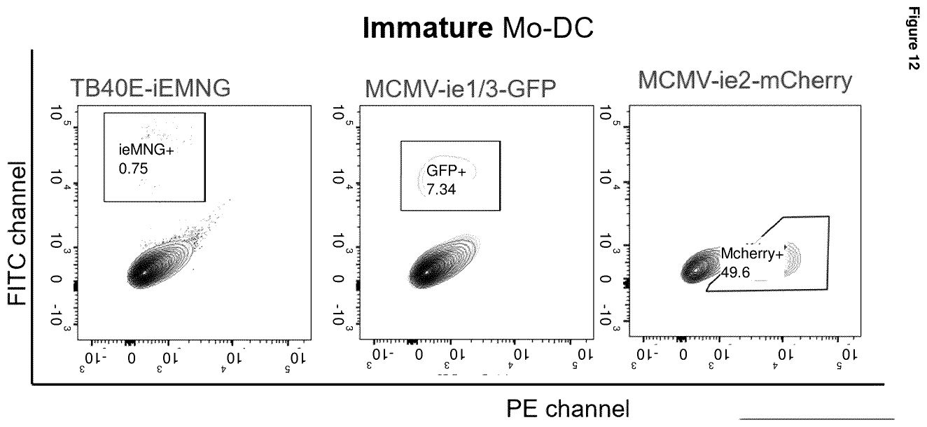

FIG. 12: Immature monocyte dendritic cells are susceptible to MCMV infection.

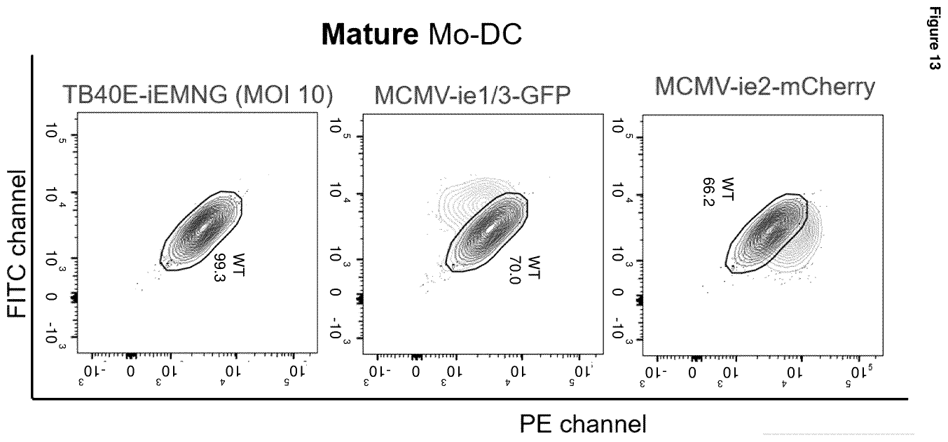

FIG. 13: Mature monocyte dendritic cells are susceptible to MCMV infection.

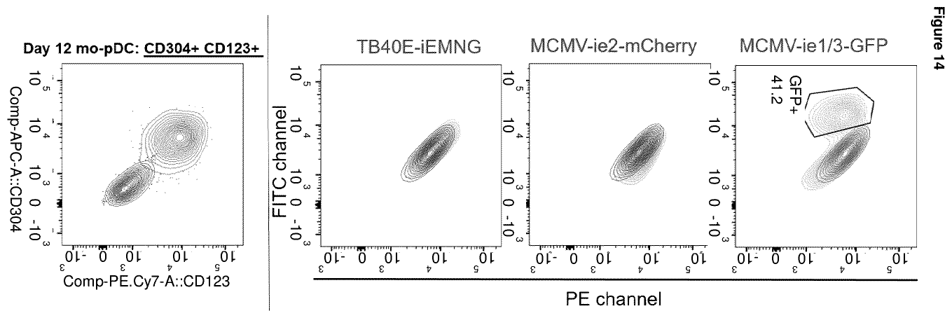

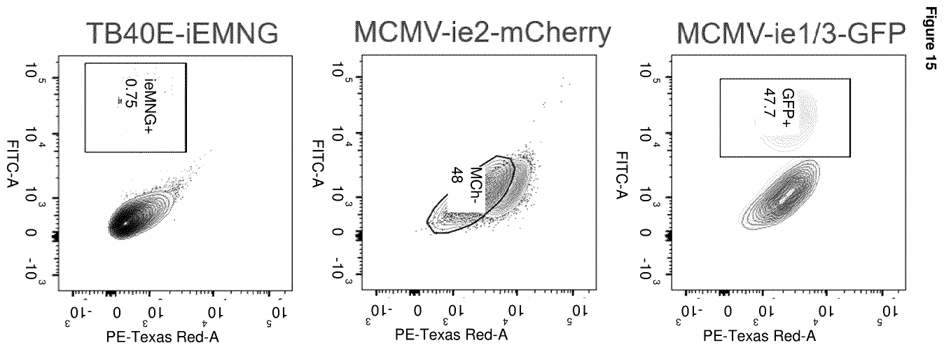

FIG. 14: Monocyte dendritic cells are susceptible to MCMV infection.

FIG. 15: M2 macrophages are susceptible to MCM infection.

FIG. 16: Long-term immune response to coronavirus variants after MCMV^{S} immunization.

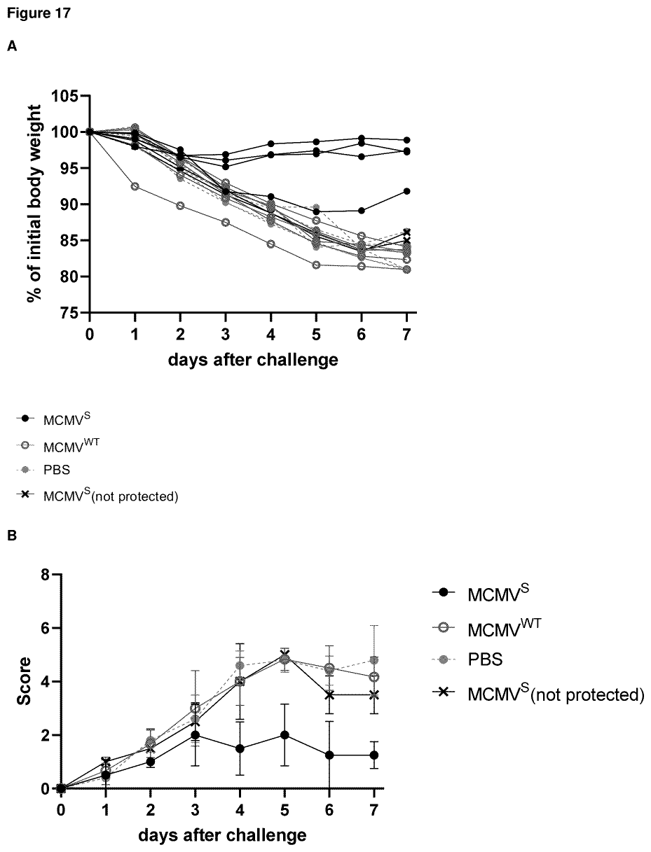

FIG. 17: Daily body weight loss and clinical scores after the viral challenge with D614G strain.

### DETAILED DESCRIPTION OF THE FIGURES

FIG. 1: Different kinds of functional antigen presenting cells induced from human monocyte by different cytokines are infected with MCMV. (A) Human monocytes were isolated from healthy donors. Different cytokines were used to induce differentiation into different kinds of functional antigen presenting cells. (B) A recombinant MCMV carrying a GFP reporter under the control of ie1/3 (immediate early) promoter and a mCherry in control of scp (late) promoter. Monocyte derived classical dendritic cells were obtained and infected with MCMV. Early and late gene vectored transgenes were expressed. (C) Monocyte derived Plasmacytoid like derived DC were obtained. Early and late gene vectored transgenes were expressed. Strong cytokine responses were found after MCMV infection. (D) Monocyte derived Langerhans like cells were obtained. Langerin expression and phenotype correspond to primary Langerhans cells. Early and late gene vectored transgenes were expressed.

FIG. 2: MCMV ability to replicate in human cells. 6 Cell lines tested for MCMV replication. Steady decline of infectious units in supernatant. Results do not show any virus growth in any human cell line.

FIG. 3: Retinal pigment epithelial cells are permissive to MCMVs. MCMV wild-type virus grew on two independent cell lines based on retinal pigment epithelium: (A) RPE1_NEW ATCC 121119 (B) ARPE19_121119. This was in stark contrast to a previous report that MCMV does not grow on RPE-I, unless a viral gene (M112/M113) adapts to this allow growth on this cell type (Schumacher, U., et al. mutations in the M112/M113-coding region facilitate murine cytomegalovirus replication in human cells (2010) Journal of Virology, 84 (16), pp. 7994-8006. DOI: 10.1128/JVI.02624-09). We tested the virus that was used in the paper cited above (MCMVC3X-GFP) and recapitulated their finding. We considered that two genes were potentially different between our MCMVdFP and their MCMVC3X-GFP: m129/m131, also known as MCK-2 (mutated in the C3X clone) and IE2 (due to GFP insertion).

FIGS. 4: M129 and m131 mutants do not explain RPE growth phenomenon. Hence, these genes are not required for growth in RPE cells. C3X has a frameshift mutation that mutates virus ORFs m129 and m131. Two independent virus mutants were tested with such a mutation for growth in RPE-I cells. The parental C3X clone (w/o GFP) is shown on the right and was able to grow.

We generated a virus with a targeted frameshift mutation in the m129/m131 gene to match the mutation in the C3X clone, and with GFP expressed outside of IE2. We compared its growth to the parental virus expressing the GFP in the same location and no frame shift. Both viruses were able to grow in RPE-I with very similar kinetics. Therefore, mutation in m129/m131 is not the cause of the growth defect in RPE-I.

FIG. 5: The growth potential of RPE cells infected different MCMV mutants, i.e. c3x mutant and IE2 mutant. The MCMVC3X-GFP was deep-sequenced, revealing a deletion in the non-coding region of the exon 1 of IE2. The deletion included the TATA box of this gene. Hence, we considered that the deletion of IE2 may result in loss of MCMV replication in human RPE-I cells. This was confirmed using a mutant virus, where the genetic sequence of IE2 was disrupted.

FIG. 6: MCMV does not proliferate in MoDC. Dendritic cells derived from primary human monocytes were tested for MCMV replication. A steady decline of infectious units was observed in supernatant. Results did not show any virus growth in any human cell line but in mouse fibroblasts tested in this screening.

FIG. 7: Antigen-specific CD8 responses are stronger to cells infected with MCMV than HCMV. (A) schema of experiment steps. (B) and (C) *NLVPMVATV is an immunodominant antigenic peptide derived from the pp65 antigen of HCMV. Hence, it is present in transgenic MCMV-NLV and in the HCMV clones used. HCMV-TB40E lacks US2, US3, US6-immune evasive genes downregulating HLA-I expression on virus infected cells, and thus CD8 T cell recognition of infected cells. KL7-SE is the wild-type HCMV with all genes in it.

FIG. 8: Schematic images of the recombinant MCMV vector genome (A) MCMV-S genome map. (B) The SARS-COV-2 spike ORF was inserted in place of ie2.

FIG. 9: MCMV immunized hamster weight loss and score upon SARS-COV-2 challenge. Hamsters were intraperitoneally immunized with MCMV^{S}, MCMV^{WT}, or PBS. 7 weeks after hamsters were intranasally infected with 2000 PFU of SARS-COV-2 (D614G strain). A virus with the disrupted IE2 sequence was tested for immunogenicity and immune protection in hamster. Mice vaccinated with MCMV expressing SARS-COV-2 spike instead of ie2 were challenged with SARS-COV-2 and monitored for weight loss and clinical scores. Our data argue that vaccinated hamsters were protected from disease.

FIG. 10: MCMV immunized hamster weight loss and score upon SARS-COV-2 challenge. (A) Neutralization assay with SARS-COV-2 D614G. 2 hamsters did not show any antibody responses. Neutralization assays were performed according to the published protocol (Kim et al., 2022, Cell. Mol. Immunol.). (B) and (C) Hamsters were intraperitoneally immunized with MCMV^{S}, MCMV^{WT}, or PBS. 14 weeks after hamsters were intranasally infected with 2000 PFU of SARS-CoV-2 (D614G strain). All hamsters that showed S-specific antibodies were protected from SARS-COV-2 illness. (D) no infectious SARS-COV-2 was identified in the lung homogenates of the hamsters that showed S-specific antibodies. (E) The serum titers in an ELISA assay with the indicated spike variants (Wuhan, alpha, kappa, beta) at 4 weeks post intraperitoneal immunization with a single vaccine dose of MCMV-Spike is shown.

FIG. 11: Infection with MCMV-S induces antibody response in rats. (A) Schematic representation of the recombinant MCMV vector (MCMV-S). The SARS-COV-2 Spike ORF was inserted in place of the MCMV ie2. (B) The setup of the rat experiment. (C) Wistar female rats were immunized via intraperitoneal route with 106 PFU/rat of MCMV vector expressing Spike (MCMV-S) or WT-MCMV as a control. Six weeks post immunization the blood was collected for sera analysis. Spike-specific IgG was assessed by ELISA (n=5).

FIG. 12: Immature monocyte dendritic cells are susceptible to MCMV infection. MCMV-WT shown as black background on each graph. HCMV TB40E-IEMNG, MCMV-ie2-mCherry, MCMV-ie1/3-GFP (nMOI 2) are shown as well. 24 hpi (duplicate, representative plot) was performed. Human mo-DCs were generated from monocytes isolated from healthy CMV-negative blood donors. 1000 UI/mL human GM-CSF and 1000 UI/mL human IL-4 were used for cytokine differentiation for 5 days. To measure the reporter-positive population, mo-DCs were harvested and analyzed by flow cytometry. Gating was conducted on live, single cells.

FIG. 13: Mature monocyte dendritic cells are susceptible to MCMV infection. MCMV-WT shown as black background on each graph. HCMV TB40E-iEMNG, MCMV-ie2-mCherry, MCMV-ie1/3-GFP (nMOI 2) are shown as well. 24 hpi (duplicate, representative plot) was performed. Human mo-DCs were generated from monocytes isolated from healthy CMV-negative blood donors. 1000 UI/mL human GM-CSF and 1000 UI/mL human IL-4 was used for cytokine differentiation for 5 days. To induce maturation, 200 UI/mL human TNFα, 10 ng/ml human IL-1B, 1000 UI/mL human IL-6 and 1 μg/mL PGE2 were used to stimulate mo-DCs for 24 hours.

FIG. 14: Monocyte-plasmacytoid dendritic cells are susceptible to MCMV infection. MCMV-WT shown as black background on each graph. HCMV TB40E-IEMNG, MCMV-ie2-mCherry, MCMV-ie1/3-GFP (nMOI 2) are shown as well. 24 hpi (duplicate, representative plot) was performed. Human mo-pDCs were generated from monocytes isolated from healthy CMV-negative blood donors. Cells were cultivated in medium containing Flt3-L (100 ng/ml) for 5 days and then the medium was changed to Flt3-L+ CpG-A (2336) ODN (5 μg/mL) until day 9. To verify the mo-pDC phenotype, an antibody staining with CD304 and CD123 was performed.

FIG. 15: M2 macrophages are susceptible to MCM infection. MCMV-WT shown as black background on each graph. HCMV TB40E-iEMNG, MCMV-ie2-mCherry, MCMV-ie1/3-GFP (nMOI 5) are shown as well. 24 hpi (duplicate, representative plot) was performed. Human mo-DCs were generated from monocytes isolated from healthy CMV-negative blood donors. 20 ng/ml human M-CSF was used for cytokine differentiation for 5 days. Subsequently, 1000 UI/ml human IL-4, 10 UI/ml human IL-13, 1000 UI/ml human IL-6 were used to stimulate the cells for 24 hours.

FIG. 16: long-term immune response to coronavirus variants after MCMV^{S} immunization. (A) Schematic image of the experimental setup of the long-term challenge experiment. (B) The pseudotyped virus neutralizing titers (pVNT50) against the Delta and the Omicron variants. Each symbol indicates the following: closed for the short-term vaccinated cohort and open for the long-term vaccinated cohort. (C-D) The pVNT50 values of individual hamsters 4 weeks or 3 months after vaccination against (C) the Delta variant or (D) the Omicron variant. (E) The 3-month time-course kinetics of neutralizing antibody responses against the live D614G variant. Each symbol indicates an individual hamster and X-marked hamsters indicated the unprotected hamsters upon the viral challenge.

FIG. 17: Daily body weight loss (A) and clinical scores (B) after the viral challenge with D614G strain.

### EXAMPLES

The following examples are particular embodiments of the invention without being limiting in scope.

As shown herein, a mouse cytomegalovirus (MCMV) vaccine vector platform elicits a robust immune response in non-mouse, such or non-murine, species. The MCMV is considered strictly species specific, with extremely limited replication in cells that do not belong to its natural host species. We comprehensively tested the MCMV growth in human cells and identified that (contrary to reports in literature) it is able to be grown in human retinal pigment epithelium (RPE).

We identified a viral gene that is essential for MCMV replication in RPE, but irrelevant for its growth in murine cells. The gene in question is called immediate-early 2 (ie2). We generated recombinant viruses lacking ie2 and validated that they do not grow in RPE cells. Thus, we have generated an MCMV that is naturally capable of growing in murine cells but acts as a single-cycle safe viral vector in non-mouse/non-murine/human cells.

Our approach is qualitatively different from published evidence and enables MCMV-based vaccine vectors against infectious pathogens. As an example of the vaccine efficacy and suitability, on the paradigmatic examples of an HCMV antigen encoded by the MCMV vector and of a Coronavirus antigen tested in vivo, are shown below. We show that the vector eliminates biosafety concerns posed by the MCMV ability to grow in human retinal epithelial cells.

### Example 1: Inducing Human Monocyte to Differentiate to Different Kinds of Functional Antigen Presenting Cells with Different Cytokines and Infecting them with MCMV

Different kinds of functional antigen presenting cells induced from human monocyte by different cytokines are infected with MCMV. Human monocytes were isolated from healthy donors.

Different cytokines were used to induce differentiation into different kinds of functional antigen presenting cells (FIG. 1A). A recombinant MCMV carrying a GFP reporter under the control of ie1/3 (immediate early) promoter and a mCherry in control of scp (late) promoter. Monocyte derived classical dendritic cells were obtained and infected with MCMV. Early and late gene vectored transgenes were expressed (FIG. 1B). Monocyte derived Plasmacytoid like derived DC were obtained. Early and late gene vectored transgenes were expressed. Strong cytokine responses were found after MCMV infection (FIG. 1C). Monocyte derived Langerhans like cells were obtained. Langerin expression and phenotype correspond to primary Langerhans cells. Early and late gene vectored transgenes were expressed (FIG. 1D).

### Example 2: Testing MCMV Ability to Replicate in Human Cells

The steps according to the following protocol were performed:
1) One day before the infection 40k cells were seeded 2) Infection with MCMV^{WT} at an MOI 1 for an hour at 37 3) PBS washing twice and new culture medium 4) Infected cells were incubated at 37 5) SN uptake at the indicated time points 6) Titration of infectious virus on MEF cells. As shown in FIG. 2, 6 Cell lines tested for MCMV replication. Steady decline of infectious units in supernatant. Results do not show any virus growth in any human cell line.

### Example 3: Retinal Pigment Epithelial Cells are Permissive to MCMVs

MCMV wild-type virus grew on two independent cell lines based on retinal pigment epithelium RPE1_NEW ATCC 121119 (FIG. 3A), ARPE19_121119 (FIG. 3B). This was in stark contrast to a previous report that MCMV does not grow on RPE-I, unless a viral gene (M112/M113) adapts to this allow growth on this cell type (Schumacher, U., et al. mutations in the M112/M113-coding region facilitate murine cytomegalovirus replication in human cells (2010) Journal of Virology, 84 (16), pp. 7994-8006. DOI: 10.1128/JVI.02624-09). We tested the virus that was used in the paper cited above (MCMVC3X-GFP) and recapitulated their finding. We considered that two genes were potentially different between our MCMVdFP and their MCMVC3X-GFP: m129/m131, also known as MCK-2 (mutated in the C3X clone) and IE2 (due to GFP insertion).

### Example 4: M129 and m131 are not Required for Growth in RPE Cells

We generated a virus with a targeted frameshift mutation in the m129/m131 gene to match the mutation in the C3X clone, and with GFP expressed outside of IE2. We compared its growth to the parental virus expressing the GFP in the same location and no frame shift. Both viruses were able to grow in RPE-I with very similar kinetics. Therefore, mutation in m129/m131 is not the cause of the growth defect in RPE-I. Hence, these genes are not required for growth in RPE cells. C3X has a frameshift mutation that mutates virus ORFs m129 and m131. Two independent virus mutants were tested with such a mutation for growth in RPE-I cells. The parental C3X clone (w/o GFP) is shown in FIG. 4A and was able to grow.

### Example 5: The Phenomenon of RPE Cells Infected with MCMV^{C3X-GFP} and MCMV^{IE2KO}

The growth potential of RPE cells infected different MCMV mutants, i.e. c3x mutant and IE2 mutant. The MCMV^{C3X-GFP} was deep-sequenced, revealing a deletion in the non-coding region of the exon 1 of IE2. The deletion included the TATA box of this gene. Hence, we considered that the deletion of IE2 may result in loss of MCMV replication in human RPE-I cells. This was confirmed using a mutant virus, where the genetic sequence of IE2 was disrupted.

ie2 gene sequence (SEQ ID NO: 1):
ccgtgctgattcatatgccatatgagtgtattagggggctttccgcttgg
gaaattgggtaaaaagtccccgtattactcacatagggggcgtttggctt
tgcaaattaggggatttcagtgcatttggcattaaaaactattggttcta
gtcataaaacgggcggagttAACCATATAAAAGCTGTCCCCCATGCCATT
CGAGCCCAGAGCAGGACGGACCGCGGCTCGATACGACCCTATCTACGTTa
acgagcgggtgaacggagccccccaggccgggtaaggttgcattgggctc
ggctatgggacggggatttggcccggtactttaatatgggtggggtct

The sequence in capitals is the deleted sequence in the 5′ region of the IE2 gene in C3X-GFP MCMV. The bold type sequence is TATA box sequence. The underlined sequence is exon 1.

### Example 6: MCMV does not Proliferate in Dendritic Cells Derived from Primary Human Monocytes

Dendritic cells derived from primary human monocytes were tested for MCMV replication. As shown in FIG. 6, a steady decline of infectious units was observed in supernatant. Results did not show any virus growth in any human cell line but in mouse fibroblasts tested in this screening.

### Example 7: Antigen-Specific CD8 Responses are Stronger to Cells Infected with MCMV than HCMV

The schema of experiment steps is shown in FIG. 1A. *NLVPMVATV is an immunodominant antigenic peptide derived from the pp65 antigen of HCMV. Hence, it was present in transgenic MCMV-NLV and in the HCMV clones used. HCMV-TB40E lacks US2, US3, US6-immune evasive genes downregulating HLA-I expression on virus infected cells, and thus CD8 T cell recognition of infected cells. KL7-SE is the wild-type HCMV with all genes in it (FIGS. 7B and 7C).

### Example 8: MCMV Immunized Hamster Weight Loss and Score Upon SARS-COV-2 Challenge

Hamsters were intraperitoneally immunized with MCMV^{S}, MCMV^{WT}, or PBS. 7 weeks after hamsters were intranasally infected with 2000 PFU of SARS-COV-2 (D614G strain). A virus with the disrupted IE2 sequence was tested for immunogenicity and immune protection in hamster. Mice vaccinated with MCMV expressing SARS-COV-2 spike instead of ie2 were challenged with SARS-COV-2 and monitored for weight loss and clinical scores (FIGS. 9A and 9B). Our data argue that vaccinated hamster were protected from disease.

### Example 9: MCMV Immunized Hamster Weight Loss and Score Upon SARS-COV-2 Challenge

Neutralization assay with SARS-COV-2 D614G. 2 hamsters did not show any antibody responses (FIG. 10A). Neutralization assays were performed according to the published protocol (Kim et al., 2022, Cell. Mol. Immunol.). Hamsters were intraperitoneally immunized with MCMV^{S}, MCMV^{WT}, or PBS. 14 weeks after hamsters were intranasally infected with 2000 PFU of SARS-CoV-2 (D614G strain). All hamsters that showed S-specific antibodies were protected from SARS-COV-2 illness (FIGS. 10B and 10C). No infectious SARS-COV-2 was identified in the lung homogenates of the hamsters that showed S-specific antibodies (FIG. 10D). The serum titers in an ELISA assay with the indicated spike variants (Wuhan, alpha, kappa, beta) at 4 weeks post intraperitoneal immunization with a single vaccine dose of MCMV-Spike is shown (FIG. 10E).

### Example 10: Infection with MCMV-S Induces Antibody Response in Rats

To explore the potential of MCMV-S vectors to be used in cross-species immunization, rats were immunized, and their blood was collected for sera analysis. MCMV as species-specific virus can infect rats, but it cannot fully replicate causing abortive infection, so the aim of this experiment was to see if the infection could lead to antibody response to vectored antigen. Shown here are the data from terminal time-point (42 dpi). All rats showed antibody response SARS-COV-2 S protein.

Schematic representation of the recombinant MCMV vector (MCMV-S) is shown in FIG. 11A. The SARS-COV-2 Spike ORF was inserted in place of the MCMV ie2. The setup of the rat experiment is shown in FIG. 11B. Wistar female rats were immunized via intraperitoneal route with 106 PFU/rat of MCMV vector expressing Spike (MCMV-S) or WT-MCMV as a control. Six weeks post immunization the blood was collected for sera analysis. Spike-specific IgG was assessed by ELISA (n=5) (FIG. 11C).

### Example 11: Susceptibility of Human Monocyte to MCMV Infection

To explore the potential of MCMV vectors to infect human cells and induce the expression of exogenous antigens in antigen-presenting cells, human monocyte-derived cells were generated from healthy CMV-negative human blood donors. After monocyte isolation by magnetic sorting, immature Mo-DC, mature Mo-DC, mo-plasmacytoid DC (mo-pDC) and M2 macrophages were obtained by cytokine stimulation. For immature moDCs, 1000 UI/mL human GM-CSF and 1000 UI/mL human IL-4 were used for 5 days. For mature moDCs, an additional stimulation with 200 UI/mL human TNFα, 10 ng/ml human IL-1β, 1000 UI/mL human IL-6 and 1 μg/mL PGE2 were used for 24 hours. For mo-PDCs, cells were cultivated in medium containing Flt3-L (100 ng/ml) for 5 days and then the medium was changed to Flt3-L+ CpG-A (2336) ODN (5 μg/mL) until day 9. For M2 macrophages, 20 ng/mL human M-CSF was used for cytokine differentiation for 5 days, with an additional 24 hour stimulation with media containing 1000 UI/mL human IL-4, 10 UI/ml human IL-13 and 1000 UI/mL human IL-6. Then, cells were infected with MCMV reporters that allow the monitoring of fluorescent protein expression driven by endogenous promoters. Immature Mo-DC, mature Mo-DC and M2 macrophages were infected with human CMV (HCMV TB40E-iEMNG) and Murine CMV (MCMV-ie2-mCherry, MCMV-ie1/3-GFP). As shown in FIG. 12-15, all of them were susceptible to MCMV infection.

### Example 12: Long-Term Immune Response to Coronavirus Variants after MCMV^{S} Immunization

### In Vivo Infection:

Syrian hamsters (8-9 weeks old, male) were i.p. immunized with 106 PFU of recombinant MCMVS or MCMVWT diluted in PBS, or PBS (200 μL per animal). Blood was acquired at indicated time points to control the immune status of the vaccinated animals.

After the indicated time points, challenge experiments were performed. Vaccinated hamsters were anesthetized with ketamine (100 mg/kg) and Medetomidin (1 mg/mL) solution. Thereafter, anesthetized animals were intranasally infected with 103 PFU of SARS-COV-2 (50 μL per nasal cavity). 45 min after anesthesia, asleep hamsters were wakened by administering 100 μL atipamezole (1.7 mg/kg) in NaCl solution. All infected hamsters were monitored for the development of virus-associated symptoms and scored daily.

As can be derived from FIG. 16, in particular FIGS. 16C and D, the pVNT50 values of individual hamsters 4 weeks or 3 months after vaccination against (C) the Delta variant or (D) the Omicron variant show that increased antibody titers are obtained over time. This represents a beneficial and unexpected result, indicating that antigen-specific immunity may, after administration of a single dose of the vaccine mCMV vector, not wane or reduce over time, but rather increase, thus reducing the need for booster immunizations, as is the case for e.g., mRNA vaccines.

### In Vitro Virus Neutralization Titer (VNT) Measurement:

The serum neutralization assay was performed as described previously (Y. Kim et al., 2022). Briefly, sera were heat-inactivated sera at 56° C. for 30 min. Thereupon, sera were serially diluted at 1:2 steps, followed by incubation with SARS-COV-2 (1,000 PFU/mL) for an hour at RT. Vero-E6 cells (at the density of 2×10⁴/well in 96-well plates) were inoculated with serum/virus mixture for 1 h. After the inoculum removal, subsequent procedures were the same as the plaque assay. Serum titer that results in a 50% reduction of virus plaques (VNT50) was analyzed by nonlinear regression analysis by GraphPad Prism.

Pseudovirus Neutralization Titer (pVNT) Measurement:

Pseudovirus neutralization assays were performed as described in the previous publications (Hoffmann et al., 2021; Y. Kim et al., 2022). For neutralization experiments, pseudotype particles and serum dilution were mixed 1:1 ratio and incubated for 60 min at 37° C. before being inoculated onto VeroE6 cells grown in 96-well plates. At 22-24 h post-infection, GFP expression was measured by using Incucyte S3 (Sartorius, Goettingen, Germany). Similar to the VNT50, serum titer that results in a 50% reduction of GFP signal (pVTN50) was analyzed by nonlinear regression analysis by GraphPad Prism.

To the knowledge of the inventor, the data show for the first time ever that a CMV based vaccine vector provides stable and lasting humoral immunity in a species that is not the natural host of the virus, thus demonstrating in a proof of principle that MCMV vectors might be used as vehicles for antigens targeting human pathogens in clinical settings.

1. A method for inducing an antigen-specific immune response in a subject, comprising administering to said subject a replication-deficient murine Cytomegalovirus (MCMV) vector, wherein the subject is not a mouse, and wherein said vector expresses a disease antigen.

2. The method according to claim 1, wherein an open reading frame for the disease antigen is inserted in the vector to replace any non-essential viral genomic sequences.

3. The method according to claim 1, wherein the vector has a disrupted immediate-early 2 (ie2) gene, causing a replication deficiency of said vector in a non-mouse subject.

4. The method according to claim 1, wherein the ie2 gene is disrupted by deletion of the ie2 gene or part thereof.

5. The method according to claim 4, wherein an open reading frame for the disease antigen is inserted in the vector to replace the ie2 open reading frame or part thereof.

6. The method according to claim 1, wherein the open reading frame for the disease antigen is expressed by an immediate-early MCMV promoter, a constitutive promoter or an inducible MCMV promoter.

7. The method according to claim 1, wherein the subject is a human.

8. The method according to claim 1, wherein said immune response comprises a neutralising antibody response or a T-cell response against the disease antigen, or priming, maintaining and/or boosting the immune system of the subject.

9. The method according to claim 1, wherein the disease antigen comprises an antigen or fragment(s) thereof of a pathogenic virus.

10. The method according to claim 9, wherein the viral antigen comprises an antigen selected from the group consist of a coronavirus, influenza virus, herpes virus, polyoma virus, papilloma virus, orthomyxovirus, paramyxovirus, picornavirus, parvovirus, reovirus, rhabdovirus, togavirus, African swine fever, adenovirus, coxsackie, arenavirus, a retrovirus, alfavirus, pestivirus, a flavivirus and an arbovirus antigen.

11. The method according to claim 1, wherein the disease antigen is:
a. a bacterial antigen, or
b. a parasitic antigen.

12. The method according to claim 1, wherein a pharmaceutical composition comprising the replication-deficient murine Cytomegalovirus (MCMV) vector is administered to the subject, wherein said vector is suitable to induce an antigen-specific immune response in a subject, wherein said vector expresses a disease antigen, and wherein the composition is configured for administration to a non-mouse subject, and comprises a human or veterinary acceptable carrier, excipient, vehicle and/or adjuvant.

13. (canceled)

14. The method according to claim 12, wherein the pharmaceutical composition is a vaccine.

15. (canceled)

16. The method according to claim 4, wherein the ie2 gene is disrupted by deletion of a TATA box or deletion of the ie2 open reading frame or part thereof.

17. The method according to claim 11, wherein the disease antigen is a bacterial antigen, and comprising an antigen from a Mycobacterium, Listeria, Chlamydia, Rickettsia, Yersinia, Helicobacter, Legionella, Streptococcus, Staphylococcus, Neisseria, or Salmonella.

18. The method according to claim 11, wherein the disease antigen is parasitic antigen, and comprises an antigen from a plasmodium or trypanosome.

19. The method according to claim 1, wherein the subject is non-human animal.

20. The method according to claim 1, wherein the disease antigen comprises an antigen or fragment(s) thereof of a cancer cell.

21. The method according to claim 10, wherein the viral antigen is a Spike, Envelope, Membrane and/or Nucleoprotein of SARS-COV-2 or any part thereof.


SEQUENCE LISTING (4 sequences)

SEQ ID NO: 1 - DNA, 348 bp, synthetic construct
  misc_feature 1..348: note = ie2 gene
  source 1..348: mol_type = other DNA
ccgtgctgattcatatgccatatgagtgtattagggggctttccgcttgggaaattgggtaaaaagtccccgtattactcacatagggggcgtttggctttgcaaattaggggatttcagtgcatttggcattaaaaactattggttctagtcataaaacgggcggagttaaccatataaaagctgtcccccatgccattcgagcccagagcaggacggaccgcggctcgatacgaccctatctacgttaacgagcgggtgaacggagccccccaggccgggtaaggttgcattgggctcggctatgggacggggatttggcccggtactttaatatgggtggggtct

SEQ ID NO: 2 - DNA, 3822 bp, synthetic construct
  misc_feature 1..3822: note = Nucleic sequence encoding a spike protein of the SARS-CoV-2
  source 1..3822: mol_type = other DNA
atgttcgtgtttctggtgctgctgcctctggtgtccagccagtgtgtgaacctgaccacaagaacccagctgcctccagcctacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctgggcgtctactatcacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcaacctcgtgcgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaaccttcctgctgaagtacaacgagaacggcaccatcaccgacgccgtggattgtgcccttgatcctctgagcgagacaaagtgcaccctgaagtccttcaccgtggaaaagggcatctaccagaccagcaacttccgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaagatcgccgactacaactacaagctgcccgacgacttcaccggctgtgtgattgcctggaacagcaacaacctggactccaaagtcggcggcaactacaattacctgtaccggctgttccggaagtccaatctgaagcccttcgagcgggacatctccaccgagatctatcaggccggcagcaccccttgtaacggcgtggaaggcttcaactgctacttcccactgcagtcctacggctttcagcccacaaatggcgtgggctatcagccctacagagtggtggtgctgagcttcgaactgctgcatgcccctgccacagtgtgcggccctaagaaaagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccaggacgtgaactgtaccgaagtgcccgtggccattcacgccgatcagctgacacctacatggcgggtgtactccaccggcagcaatgtgtttcagaccagagccggctgtctgatcggagccgagcacgtgaacaatagctacgagtgcgacatccccatcggcgctggcatctgtgccagctaccagacacagacaaacagccccagacgggccagatctgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaatagagccctgacagggatcgccgtggaacaggacaagaacacccaagaggtgttcgcccaagtgaagcagatctacaagacccctcctatcaaggacttcggcggcttcaatttcagccagattctgcccgatcctagcaagcccagcaagcggagcttcatcgaggacctgctgttcaacaaagtgacactggccgacgccggcttcatcaagcagtatggcgattgtctgggcgacattgccgccagggatctgatttgcgcccagaagtttaacggactgacagtgctgccaccactgctgaccgatgagatgatcgcccagtacacatctgccctgctggccggcacaatcacaagcggctggacatttggagctggcgccgctctgcagatcccctttgctatgcagatggcctaccggttcaacggcatcggagtgacccagaatgtgctgtacgagaaccagaagctgatcgccaaccagttcaacagcgccatcggcaagatccaggacagcctgagcagcacagcaagcgccctgggaaagctgcaggacgtggtcaaccagaatgcccaggcactgaacaccctggtcaagcagctgtcctccaacttcggcgccatcagctctgtgctgaacgatatcctgagcagactggacaaggtggaagccgaggtgcagatcgacagactgatcaccggaaggctgcagtccctgcagacctacgttacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgacccagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggataagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggccctggtacatctggctgggctttatcgccggactgattgccatcgtgatggtcacaatcatgctgtgttgcatgaccagctgctgtagctgcctgaagggctgttgtagctgtggcagctgctgcaagttcgacgaggacgattctgagcccgtgctgaagggcgtgaaactgcactacacctaa

SEQ ID NO: 3 - DNA, 79 bp, synthetic construct
  misc_feature 1..79: note = Deleted Sequence in IE2
  source 1..79: mol_type = other DNA
aaccatataaaagctgtcccccatgccattcgagcccagagcaggacggaccgcggctcgatacgaccctatctacgtt

SEQ ID NO: 4 - DNA, 46 bp, synthetic construct
  misc_feature 1..46: note = Exon 1 of ie2 gene
  source 1..46: mol_type = other DNA
gcccagagcaggacggaccgcggctcgatacgaccctatctacgtt